(19) United States
(12) Patent Application Publication
(10) Pub. No.: US 2025/0367238 A1
(43) Pub. Date: Dec. 4, 2025

(54) SELECTED RENAL CELL POPULATIONS, CHARACTERISTICS AND USES THEREOF

(71) Applicant: Pro Kidney, Grand Cayman (KY)

(72) Inventors: Prakash Narayan, Winston-Salem, NC (US); Timothy A. Bertram, Grand Cayman (KY); Deepak Jain, Winston-Salem, NC (US)

(21) Appl. No.: 18/876,597

(22) PCT Filed: Jun. 29, 2023
(86) PCT No.: PCT/US2023/069391

§ 371 Date: Dec. 18, 2024

Related U.S. Application Data
(60) Provisional application No. 63/356,798, filed on Jun. 29, 2022.

(51) Int. Cl. A61K 35/22

(52) U.S. Cl. CPC A61K 35/22

(57) ABSTRACT

Methods of identifying an enriched heterogeneous renal cell population having therapeutic potential, enriched heterogeneous renal cell populations having therapeutic potential and uses for same.

### BACKGROUND

Chronic kidney disease (CKD) is reaching, or has reached, pandemic proportions (Thurlow J S, et al., Am J Nephrol 52 (2021): 98-107; Murphy D, et al., Intern Med 65 (2016): 473-481; Hill N R, et al., PLOS One 11:e0158765.doi: 10.1371/journal.pone.0158765). Prevalence data from the U.S. to Europe show that approximately 10% of the general population has stage 1-3 CKD (ERA, 2009; USRDS, 2011; Jha et al., Lancet. 382 (2013): 260-72). CKD has increased >33% between 1996 and 2006 in the US alone (U.S. Renal Data System. Costs of CKD and ESRD. Minneapolis, MN, 2007). Meanwhile, management of CKD remains a challenge for nephrologists. Most standard of care treatments for CKD are small molecules targeting biochemical pathways in the kidney to affect single or related co-morbidities. However, these treatments do not affect the underlying CKD kidney's glomerular and tubulointerstitial dysfunction. Ultimately, in patients that progress to end stage renal disease (ESRD), renal replacement therapy (RRT; dialysis or transplantation) is required for survival. There remains an unmet need for a therapeutic modality that directly addresses the diseased kidney's tissue biology. Such a therapeutic modality can potentially arrest or even reverse CKD progression and mitigate or avert the need for RRT.

Selected renal cells (SRC), a heterogeneous renal cell population enriched for renal epithelial cells, is being advanced as autologous cell-based therapy for treatment of CKD (Stavas J, et al., Am J Nephrol 53 (2022): 50-58.; Stavas J, et al., KI Reports, in press). Derived from the donor kidney, SRC is an admixture principally of proximal tubular epithelial cells (Presnell S C, et al, Tissue Eng Part C Methods 17 (2011): 261-73). Results from a Phase II clinical trial in a diabetic kidney disease cohort suggested that SRC administration is safe and is accompanied by improvement in renal function (Stavas J, et al., KI Reports, in press).

There is a need in the art to identify heterogenous renal cell populations, such as SRCs, capable of improving renal function in CKD patients, and of mitigating, or averting, the need for RRT.

### BRIEF SUMMARY

The present disclosure describes a method of identifying an enriched heterogeneous renal cell population as having therapeutic potential. In the method, it is determined whether cells of the enriched heterogeneous renal cell population express (i) NEPH1 and (ii) one or more markers, where the one or more markers may be any one or more of nephrin (NPHS1), podocin (NPHS2), homeobox protein sine oculis homeobox homolog 2 (SIX2), odd-skipped related 1 (OSR1), lim homeobox protein 1 (LHX1), and fibroblast growth factor 8 (FGF8), receptor for activated C kinase 1 (RACK1) or rearranged during transfection (RET). The enriched heterogeneous renal cell population is identified as having a therapeutic potential if cells of the enriched heterogeneous renal cell population express (i) NEPH1 and (ii) the one or more markers.

### BRIEF DESCRIPTION OF FIGURES

FIG. 1A-1B: Depiction of co-expression interactome analysis output by STRING (FIG. 1A) and Genemania (FIG. 1B) across human tissue for eight nodes expressed on an enriched heterogeneous renal cell population, e.g., human selected renal cells (SRCs); eight nodes=Six2, Osr1, Lhx1, Ret, Fgf8, Rack1, Nphs1, Nphs2, ret and rack1 gnb211; neither interactome analysis found nodes ret and rack1 gnb211 to be co-expressed with nodes Six2, Osr1, Lhx1, Ret, Fgf8, Rack1, Nphs1 and Nphs2 in human tissue.

FIG. 2A-2H: Provides Human Fetal Kidney Atlas generated violin plots reporting expression six2 (FIG. 2A), osr1 (FIG. 2B), Ihx1 (FIG. 2C), ret (FIG. 2D), fgf8 (FIG. 2E), rack1 (FIG. 2F), nphs1 (FIG. 2G) and nphs2 (FIG. 2H) across multiple cell types involved in kidney development. Kidney cell types: NPCa, nephron progenitor subtype a; NPCb, nephron progenitor subtype b; NPCc, nephron progenitor subtype c; NPCd, nephron progenitor subtype d; PTA, pre-tubular aggregate cells, RVCSBa, renal vesicle/comma shaped body cells subtype a; RVCSBb, renal vesicle/comma shaped body cells subtype b; SSBpr, s-shaped body cells proximal; SSBm/d, s-shaped body cells medial/distal, SSBpod, s-shaped body cells podocyte progenitors; CnT, connecting tubule cells; DTLH, distal tubule/loop of Henle cells; ErPrT, early proximal tubule cells; Pod, podocytes; UBCD, ureteric bud/collecting duct cells, IPC, interstitial progenitor cells; ICa, interstitial cells subtype a, ICb interstitial cells subtype b; Mes, mesangial cells; End, endothelial cells; Leu, leukocytes; Prolif, proliferating cells.

FIG. 3: Provides a human SRC interactome built by mapping interactions across eight nodes co-expressed by human SRCs. In addition to the eight nodes co-expressed by SRCs, the SRC interactome potentially included other nodes, such as Kirre like Nephrin Family Adhesion Molecule 1 (kirrel 1, whose expression product is NEPH1).

FIG. 4: Provides gene ontology biological processes mapping, generated using ShinyGO, of forty pathways associated with human SRCs. Of note, the pathways are predominantly involved in neonephrogenesis.

FIG. 5A-5B: Provides biological processes gene network analyses showing the human SRC interactome's association with angiogenesis (network strength=0.45) and Na+ transport (network strength=0.74). Biological processes gene network analyses performed using Human Base.

FIG. 6: Uniform manifold approximation and projection (UMAP) to show SRC population cells that express kirrel1. Dark grey dots represent cells that express kirrel1. Light grey dots represent cells that do not express kirrel1.

### DETAILED DESCRIPTION

Described herein are methods of identifying an enriched heterogeneous renal cell population as having a therapeutic potential, a composition including the enriched heterogeneous renal cell population identified as having therapeutic potential, and methods and uses of the enriched heterogeneous renal cell population having therapeutic potential.

In the methods of identifying an enriched heterogeneous renal cell population as having a therapeutic potential, the therapeutic potential of the enriched heterogeneous renal cell population may be in the treatment a kidney disease, a tubular transport deficiency, or a glomerular filtration deficiency.

The therapeutic potential of the enriched heterogeneous renal cell population identified by the methods may be a restoration of kidney function, stabilization of kidney function, improvement in kidney function, reduction of renal fibrosis, reduction in renal inflammation, induction of tubulogenesis in a kidney, induction of nephrogenesis in kidney or induction of glomerulogenesis in a kidney of a patient in need of such treatment. If the enriched heterogeneous renal cell population is identified as having a therapeutic potential, the enriched heterogeneous renal cell population may restore mineral balance or alleviate anemia in a patient in need of such treatment. If the enriched heterogeneous renal cell population is identified as having a therapeutic potential, it may delay or prevent the need for dialysis, or it may delay or prevent the need for a kidney transplant in a patient in need of a treatment for a kidney disease.

In the methods of identifying an enriched heterogeneous renal cell population as having a therapeutic potential, it may be determined whether cells of the enriched heterogeneous renal cell population express (i) NEPH1 (known variously as KIRRELI, KIRREL, kin of IRRE like (Drosophila), Kin of IRRE-like protein 1, kirre like nephrin family adhesion molecule 1, NPHS23) and (ii) one or more markers. The one or more markers may be or include any of one or more of nephrin, podocin, homeobox protein sine oculis homeobox homolog 2 (SIX2), odd-skipped related 1 (OSR1), lim homeobox protein 1 (LHX1), fibroblast growth factor 8 (FGF8), receptor for activated C kinase 1 (RACK1) or rearranged during transfection (RET). The one or more markers may be, or may include, any one, any two, any three, any four, any five, any six, any seven, or all eight of nephrin, podocin, SIX2, OSR1, LHX1, RET, FGF8 and RACK1.

In the methods of identifying an enriched heterogeneous renal cell population as having a therapeutic potential, it may be determined whether cells of the enriched heterogeneous renal cell population express (i) NEPH1 and (ii) at least two of markers nephrin, podocin, SIX2, OSR1, LHX1, RET, FGF8 and RACK1. If the expression of at least two of the markers is determined, the at least two markers may be or may include (a) SIX2 and OSR1; (b) SIX2 and LHX1; (c) SIX2 and RET; (d) SIX2 and FGF8; (e) OSR1 and LHX1; (f) OSR1 and RET; (g) OSR1 and FGF8; (h) LHX1 and RET; (i) LHX1 and FGF8; (j) RET and FGF8; (k) nephrin and SIX2; (1) nephrin and OSR1; (m) nephrin and LHX1, (n) nephrin and RET, (o) nephrin and FGF8; (p) podocin and nephrin; (q) podocin and SIX2; (r) podocin and OSR1; (s) podocin and LHX1; (t) podocin and RET; (u) podocin and FGF8; (v) RACK1 and nephrin; (w) RACK1 and podocin; (x) RACK1 and SIX2; (y) RACK1 and OSR1; (z) RACK1 and LHX1; (aa) RACK1 and RET; or (bb) RACK1 and FGF8.

In the methods of identifying an enriched heterogeneous renal cell population as having a therapeutic potential, it may be determined whether cells of the enriched heterogeneous renal cell population express (i) NEPH1 and (ii) at least three of markers nephrin, podocin, SIX2, OSR1, LHX1, RET, FGF8 and RACK1. If the expression of at least three of the markers is determined, the at least three markers may be or may include (a) SIX2, OSR1 and LHX1; (b) SIX2, OSR1 and RET; (c) SIX2, OSR1 and FGF8; (d) SIX2, LHX1 and RET; (e) SIX2, LHX1 and FGF8; (f) SIX2, RET and FGF8; (g) OSR1, LHX1 and RET; (h) OSR1, LHX1 and FGF8; (i) OSR1, RET and FGF8; (j) LHX1, RET and FGF8; (k) nephrin, SIX2 and OSR; (1) nephrin, SIX2 and LHX1; (m) nephrin, SIX2 and RET; (n) nephrin, SIX2 and FGF8; (o) nephrin, OSR and LHX1; (p) nephrin, OSR and RET; (q) nephrin, OSR and FGF8; (r) nephrin, LHX1 and RET; (s) nephrin, LHX1 and FGF8; (t) nephrin, RET and FGF8; (u) podocin, SIX2 and OSR; (v) podocin, SIX2 and LHX1; (w) podocin, SIX2 and RET; (x) podocin, SIX2 and FGF8; (y) podocin, OSR and LHX1; (z) podocin, OSR and RET; (aa) podocin, OSR and FGF8; (bb) podocin, LHX1 and RET; (cc) podocin, LHX1 and FGF8; (dd) podocin, RET and FGF8; (ee) RACK1, SIX2 and OSR; (ff) RACK1, SIX2 and LHX1; (gg) RACK1, SIX2 and RET; (hh) RACK1, SIX2 and FGF8; (ii) RACK1, OSR and LHX1; (jj) RACK1, OSR and RET; (kk) RACK1, OSR and FGF8; (11) RACK1, LHX1 and RET; (mm) RACK1, LHX1 and FGF8; (nn) RACK1, RET and FGF8; (00) nephrin, podocin and RACK1; (pp) nephrin, podocin and SIX2; (qq) nephrin, podocin and OSR; (rr) nephrin, podocin and RET; (ss) nephrin, podocin and LHX1; (tt) nephrin, podocin and FGF8; (uu) nephrin, RACK1 and SIX2; (vv) nephrin, RACK1 and OSR; (ww) nephrin, RACK1 and RET; (xx) nephrin, RACK1 and LHX1; (yy) nephrin, RACK1 and FGF8; (zz) podocin, RACK1 and SIX2; (aaa) podocin, RACK1 and OSR; (bbb) podocin, RACK1 and RET; (ccc) podocin, RACK1 and LHX1; or (ddd) podocin, RACK1 and FGF8.

In the methods of identifying an enriched heterogeneous renal cell population as having a therapeutic potential, it may be determined whether cells of the enriched heterogeneous renal cell population express (i) NEPH1 and (ii) at least four of markers nephrin, podocin, SIX2, OSR1, LHX1, RET, FGF8 and RACK1. If the expression of at least four of the markers is determined, the at least four markers may be or may include (a) SIX2, OSR1, LHX1 and RET; (b) SIX2, OSR1, LHX1 and FGF8; (c) SIX2, LHX1, RET and FGF8; (d) SIX2, OSR1, RET and FGF8; (e) OSR1, LHX1, RET and FGF8; (f) nephrin, SIX2, OSR1 and LHX1; (g) nephrin, SIX2, OSR1, and RET; (h) nephrin, SIX2, OSR1 and FGF8; (i) nephrin, SIX2, LHX1 and RET; (j) nephrin, SIX2, LHX1 and FGF8; (k) nephrin, SIX2, RET and FGF8; (1) nephrin, OSR1, LHX1 and FGF8; (m) nephrin, OSR1, LHX1 and RET; (n) nephrin, OSR1, RET and FGF8; (o) nephrin, LHX1, RET and FGF8; (p) nephrin, SIX2, OSR1 and LHX1; (q) podocin, SIX2, OSR1, and RET; (r) podocin, SIX2, OSR1 and FGF8; (s) podocin, SIX2, LHX1 and RET; (t) podocin, SIX2, LHX1 and FGF8; (u) podocin, SIX2, RET and FGF8; (v) podocin, OSR1, LHX1 and FGF8; (w) podocin, OSR1, LHX1 and RET; (x) podocin, OSR1, RET and FGF8; (y) podocin, LHX1, RET and FGF8; (z) RACK1, SIX2, OSR1 and FGF8; (aa) RACK1, SIX2, LHX1 and RET; (bb) RACK1, SIX2, LHX1 and FGF8; (cc) RACK1, SIX2, RET and FGF8; (dd) RACK1, OSR1, LHX1 and FGF8; (ee) RACK1, OSR1, LHX1 and RET; (ff) RACK1, OSR1, RET and FGF8; (gg) RACK1, LHX1, RET and FGF8; (hh) nephrin, podocin, SIX2 and OSR1; (ii) nephrin, podocin, SIX2 and LHX1; (jj) nephrin, podocin, SIX2 and RET; (kk) nephrin, podocin, SIX2 and FGF8; (11) nephrin, podocin, OSR1 and LHX1; (mm) nephrin, podocin, OSR1 and RET; (nn) nephrin, podocin, OSR1 and FGF8; (00) nephrin, podocin, LHX1 and RET; (pp) nephrin, podocin, LHX1 and FGF8; (qq) nephrin, podocin, RET and FGF8; (rr) RACK1, podocin, SIX2 and OSR1; (ss) RACK1, podocin, SIX2 and LHX1; (tt) RACK1, podocin, SIX2 and RET; (uu) RACK1, podocin, SIX2 and FGF8; (vv) RACK1, podocin, OSR1 and LHX1; (ww) RACK1, podocin, OSR1 and RET; (xx) RACK1, podocin, OSR1 and FGF8; (yy) RACK1, podocin, LHX1 and RET; (zz) RACK1, podocin, LHX1 and FGF8; (aaa) RACK1, podocin, RET and FGF8; (bbb) RACK1, nephrin, SIX2 and OSR1; (ccc) RACK1, nephrin, SIX2 and LHX1; (ddd) RACK1, nephrin, SIX2 and RET; (eee) RACK1, nephrin, SIX2 and FGF8; (fff) RACK1, nephrin, OSR1 and LHX1; (ggg) RACK1, nephrin, OSR1 and RET; (hhh) RACK1, nephrin, OSR1 and FGF8; (iii) RACK1, nephrin, LHX1 and RET; (jjj) RACK1, nephrin, LHX1 and FGF8; (kkk) RACK1, nephrin, RET and FGF8; (111) RACK1 nephrin, podocin and SIX2; (mmm) RACK1, nephrin, podocin and RET; (nnn) RACK1, nephrin, podocin and LHX1; (000) RACK, nephrin, podocin and OSR1; or (ppp) RACK, nephrin, podocin and FGF8.

In the methods of identifying an enriched heterogeneous renal cell population as having a therapeutic potential, it may be determined whether cells of the enriched heterogeneous renal cell population express (i) NEPH1 and (ii) at least five of markers nephrin, podocin, SIX2, OSR1, LHX1, RET, FGF8 and RACK1. If the expression of at least five of the markers is determined, the at least five markers may be or may include (a) SIX2, OSR1, LHX1, RET and FGF8; (b) nephrin, SIX2, OSR1, LHX1 and RET; (c) nephrin, SIX2, OSR1, LHX1 and FGF8; (d) nephrin, SIX2, LHX1, RET and FGF8; (e) nephrin, SIX2, OSR1, RET and FGF8; (f) nephrin, OSR1, LHX1, RET and FGF8; (g) podocin, SIX2, OSR1, LHX1 and RET; (h) podocin, SIX2, OSR1, LHX1 and FGF8; (i) podocin, SIX2, LHX1, RET and FGF8; (j) podocin, SIX2, OSR1, RET and FGF8; (k) podocin, OSR1, LHX1, RET and FGF8; (1) RACK1, SIX2, OSR1, LHX1 and RET; (m) RACK1, SIX2, OSR1, LHX1 and FGF8; (n) RACK1, SIX2, LHX1, RET and FGF8; (o) RACK1, SIX2, OSR1, RET and FGF8; (p) RACK1, OSR1, LHX1, RET and FGF8; (q) nephrin, podocin, SIX2, OSR1 and LHX1; (r) nephrin, podocin, SIX2, OSR1 and RET; (s) nephrin, podocin, SIX2, OSR1 and FGF8; (t) nephrin, podocin, SIX2, LHX1 and RET; (u) nephrin, podocin, SIX2, LHX1 and FGF8; (v) nephrin, podocin, SIX2, RET and FGF8; (w) nephrin, podocin, OSR1, LHX1 and RET; (x) nephrin, podocin, OSR1, LHX1 and FGF8; (y) nephrin, podocin, OSR1, RET and FGF8; (z) nephrin, podocin, LHX1, RET and FGF8; (aa) nephrin, RACK1, SIX2, OSR1 and LHX1; (bb) nephrin, RACK1, SIX2, OSR1 and RET; (cc) nephrin, RACK1, SIX2, OSR1 and FGF8; (dd) nephrin, RACK1, SIX2, LHX1 and RET; (ee) nephrin, RACK1, SIX2, LHX1 and FGF8; (ff) nephrin, RACK1, SIX2, RET and FGF8; (gg) nephrin, RACK1, OSR1, LHX1 and RET; (hh) nephrin, RACK1, OSR1, LHX1 and FGF8; (ii) nephrin, RACK1, OSR1, RET and FGF8; (jj) nephrin, RACK1, LHX1, RET and FGF8; (kk) podocin, RACK1, SIX2, OSR1 and LHX1; (11) podocin, RACK1, SIX2, OSR1 and RET; (mm) podocin, RACK1, SIX2, OSR1 and FGF8; (nn) podocin, RACK1, SIX2, LHX1 and RET; (00) podocin, RACK1, SIX2, LHX1 and FGF8; (pp) podocin, RACK1, SIX2, RET and FGF8; (qq) podocin, RACK1, OSR1, LHX1 and RET; (rr) podocin, RACK1, OSR1, LHX1 and FGF8; (ss) podocin, RACK1, OSR1, RET and FGF8; (tt) podocin, RACK1, LHX1, RET and FGF8; (uu) nephrin, podocin, RACK1, SIX2 and OSR1; (vv) nephrin, podocin, RACK1, SIX2 and LHX1; (ww) nephrin, podocin, RACK1, SIX2 and RET; (xx) nephrin, podocin, RACK1, SIX2 and FGF8; (yy) nephrin, podocin, RACK1, OSR1 and LHX1; (zz) nephrin, podocin, RACK1, OSR1 and RET; (aaa) nephrin, podocin, RACK1, OSR1 and FGF8; (bbb) nephrin, podocin, RACK1, LHX1 and RET; (ccc) nephrin, podocin, RACK1, LHX1 and FGF8; or (ddd) nephrin, podocin, RACK1, RET and FGF8.

In the methods of identifying an enriched heterogeneous renal cell population as having a therapeutic potential, it may be determined whether cells of the enriched heterogeneous renal cell population express (i) NEPH1 and (ii) at least six of markers nephrin, podocin, SIX2, OSR1, LHX1, RET, FGF8 and RACK1. If the expression of at least six of the markers is determined, the at least six markers may be or may include (a) nephrin, SIX2, OSR1, LHX1, RET and FGF8; (b) podocin, SIX2, OSR1, LHX1, RET and FGF8; (c) RACK1, SIX2, OSR1, LHX1, RET and FGF8; (d) nephrin, podocin, SIX2, OSR1, LHX1 and RET; (e) nephrin, podocin, SIX2, OSR1, LHX1 and FGF8; (f) nephrin, podocin, SIX2, LHX1, RET and FGF8; (g) nephrin, podocin, SIX2, OSR1, RET and FGF8; (h) nephrin, podocin, OSR1, LHX1, RET and FGF8; (i) nephrin, RACK1, SIX2, OSR1, LHX1 and RET; (j) nephrin, RACK1, SIX2, OSR1, LHX1 and FGF8; (k) nephrin, RACK1, SIX2, LHX1, RET and FGF8; (1) nephrin, RACK1, SIX2, OSR1, RET and FGF8; (m) nephrin, RACK1, OSR1, LHX1, RET and FGF8; (n) podocin, RACK1, SIX2, OSR1, LHX1 and RET; (o) podocin, RACK1, SIX2, OSR1, LHX1 and FGF8; (p) podocin, RACK1, SIX2, LHX1, RET and FGF8; (q) podocin, RACK1, SIX2, OSR1, RET and FGF8; (r) podocin, RACK1, OSR1, LHX1, RET and FGF8; (s) nephrin, podocin, RACK1, SIX2, OSR1 and LHX1; (t) nephrin, podocin, RACK1, SIX2, OSR1 and RET; (u) nephrin, podocin, RACK1, SIX2, OSR1 and FGF8; (v) nephrin, podocin, RACK1, SIX2, LHX1 and RET; (w) nephrin, podocin, RACK1, SIX2, LHX1 and FGF8; (x) nephrin, podocin, RACK1, SIX2, RET and FGF8; (y) nephrin, podocin, RACK1, OSR1, LHX1 and RET; (z) nephrin, podocin, RACK1, OSR1, LHX1 and FGF8; (aa) nephrin, podocin, RACK1, OSR1, RET and FGF8; or (bb) nephrin, podocin, RACK1, LHX1, RET and FGF8.

In the methods of identifying an enriched heterogeneous renal cell population as having a therapeutic potential, it may be determined whether cells of the enriched heterogeneous renal cell population express (i) NEPH1 and (ii) at least seven of markers nephrin, podocin, SIX2, OSR1, LHX1, RET, FGF8 and RACK1. If the expression of at least seven of the markers is determined, the at least seven markers may be or may include (a) podocin, RACK1, SIX2, OSR1, LHX1, RET and FGF8; (b) nephrin, RACK1, SIX2, OSR1, LHX1, RET and FGF8; (c) nephrin, podocin, SIX2, OSR1, LHX1, RET and FGF8; (d) nephrin, podocin, RACK1, OSR1, LHX1, RET and FGF8; (e) nephrin, podocin, RACK1, SIX2, LHX1, RET and FGF8; (f) nephrin, podocin, RACK1, SIX2, OSR1, RET and FGF8; (g) nephrin, podocin, RACK1, SIX2, OSR1, LHX1, and FGF8; or (h) nephrin, podocin, RACK1, SIX2, OSR1, LHX1, and RET.

In the methods of identifying an enriched heterogeneous renal cell population as having a therapeutic potential, it may be determined whether cells of the enriched heterogeneous renal cell population express (i) NEPH1 and (ii) markers nephrin, podocin, SIX2, OSR1, LHX1, RET, FGF8 and RACK1.

In the methods of identifying an enriched heterogeneous renal cell population as having therapeutic potential, determining that cells of the heterogeneous enriched renal cell population express (i) NEPH1 and (ii) the at least one (e.g., the any one, or any two, or any three, or any four, or any five, or any six, or any seven or all eight) marker(s), may identify the enriched heterogeneous renal cell population as having therapeutic potential.

In the methods of identifying an enriched heterogeneous renal cell population as having a therapeutic potential, the determining expression of (i) NEPH1 and the (ii) one or more (or at least two, or at least three, or at least four, or at least five, or at least six, or at least seven, or eight) marker(s) may further include determining percentage of cells of the enriched heterogeneous renal cell population that express (i) NEPH1 and/or the (ii) one or more (or at least two, or at least three, or at least four, or at least five, or at least six, or at least seven, or eight) marker(s). If the percentage of cells of the enriched heterogeneous renal cell population that express (i) NEPH1 and/or the (ii) one or more (or at least one, at least two, or at least three, or at least four, or at least five, or at least six, or at least seven, or eight) marker(s) is determined, then the enriched heterogeneous renal cell population may be identified as having therapeutic potential if about a certain, or particular, percentage of cells of the enriched heterogeneous renal cell population express (i) NEPH1 and/or the (ii) one or more (or at least one, or at least two, or at least three, or at least four, or at least five, or at least six, or at least seven, or eight) marker(s).

In the methods, if the percentage of cells of the enriched heterogeneous renal cell population that express (i) NEPH1 is determined, then the enriched heterogeneous renal cell population may be identified as having a therapeutic potential if between about 2.0% and about 8.0%, or between about 2.0% and about 7.5%, or between about 2.0% and about 7.0%, or between about 2.0% and about 6.5%, or between about 2.0% and about 6.0%, or between about 2.0% and about 5.5%, or between about 2.0% and about 5.0%, or between about 2.5% and about 8.0%, or between about 3.0% and about 8.0%, or between about 3.5% and about 8.0%, or between about 4.0% and about 8.0%, or between about 2.5% and about 7.5%, or between about 3.0% and about 7.0%, or between about 3.5% and about 6.5%, or between about 4.0% and about 6.0%, or between about 4.5% and about 5.5%, or between about 4.0% and about 6.5%, or between about 4.0% and about 7.0%, or between about 3.5% and about 6.5%, or between about 3.5% to about 6.0%, or about 3% to about 6.0% of cells of the enriched heterogeneous population express NEPH1.

In the methods, if the percentage of cells of the enriched heterogeneous renal cell population that express the (ii) one or more (or at least one, at least two, or at least three, or at least four, or at least five, or at least six, or at least seven, or eight) marker(s) is determined, and the one or more (at least one, or at least two, or at least three, or at least four, or at least five, or at least six, or at least seven, or eight) marker(s) is, or includes SIX2, then the enriched heterogeneous renal cell population may be identified as having therapeutic potential if at least about. 10% of cells of the enriched heterogeneous renal cell population express SIX2. Alternatively, if the one or more (at least one, or at least two, or at least three, or at least four, or at least five, or at least six, or at least seven, or eight) marker(s) is, or includes SIX2, then the enriched heterogeneous renal cell population may be identified as having therapeutic potential if at least about 0.15%, or at least about 0.20%, or at least about 0.50%, or at least about 1.0%, or at least about 1.5%, or at least about 2.0%, or at least about 2.5%, or at least about 3.0%, or at least about 3.5%, or at least about 4.0%, or at least about 4.5%, or at least about 5.0%, or at least about 5.5% of cells of the enriched heterogeneous renal cell population express SIX2. If the one or more (at least one, or at least two, or at least three, or at least four, or at least five, or at least six, or at least seven, or eight) marker(s) is, or includes SIX2, then the enriched heterogeneous renal cell population may be identified as having therapeutic potential if greater than 0% and up to at most about 35.0%, greater than 0% and up to at most about 30.0%, or greater than 0% and up to at most about 25.0%, or greater than 0% and up to at most about 20.0%, or greater than 0% and up to at most about 15.0%, or greater than 0% and up to at most about 12.5%, or greater than 0% and up to at most about 10.0%, or greater than 0% and up to at most about 7.5%, or greater than 0% and up to at most about 5.0% of the enriched heterogeneous renal cell population express SIX2. Further, if the one or more (at least one, or at least two, or at least three, or at least four, or at least five, or at least six, or at least seven, or eight) marker(s) is, or includes SIX2, then the enriched heterogeneous renal cell population may be identified as having therapeutic potential if between about 0.10% to about 15.0%, or between about 0.10% to about 10.0%, or between about 0.10% to about 8.0%, or between about 0.10% and about 6.0%, or between about 0.10% and about 4.0%, or between about 0.10% and about 2.0%, or between about 0.40% to about 15.0%, or between about 0.40% to about 10.0%, or between about 0.40% to about 8.0%, or between about 0.40% and about 6.0%, or between about 0.40% and about 4.0%, or between about 0.40% and about 2.0%, or between about 0.60% to about 15.0%, or between about 0.60% to about 10.0%, or between about 0.60% to about 8.0%, or between about 0.60% and about 6.0%, or between about 0.60% and about 4.0%, or between about 0.60% and about 2.0%, of cells of the enriched heterogeneous renal cell population express SIX2.

In the methods, if the percentage of cells of the enriched heterogeneous renal cell population that express the one or more (at least one, or at least two, or at least three, or at least four, or at least five, or at least six, or at least seven, or eight) marker(s) is determined, and the one or more (at least one, or at least two, or at least three, or at least four, or at least five, or at least six, or at least seven, or eight) marker(s) is, or includes OSR1, then the enriched heterogeneous renal cell population may be identified as having therapeutic potential if at least about 35% of cells of the enriched heterogeneous renal cell population express OSR1. Alternatively, if the one or more (at least one, or at least two, or at least three, or at least four, or at least five, or at least six, or at least seven, or eight) marker(s) is, or includes OSR1, then the enriched heterogeneous renal cell population may be identified as having therapeutic potential if at least about 45%, or at least about 47.5%, or at least about 50%, or at least about 52.5%, or at least about 55%, or at least about 57.5%, or at least about 60%, or at least about 62.5%, or at least about 65%, or at least about 67.5%, or at least about 70% or at least about 72.5%, or at least about 75% of cells of the enriched heterogeneous renal cell population express OSR1. If the one or more (at least one, or at least two, or at least three, or at least four, or at least five, or at least six, or at least seven, or eight) marker(s) is, or includes OSR1, then the enriched heterogeneous renal cell population may be identified as having therapeutic potential if between about 35% and about 99%, or between about 35% and about 98%, or between about 35% and about 96%, or between about 35% and about 94%, or between about 35% and about 92%, or between about 35% and about 90%, or between 40% and 99%, or between about 40% and about 98%, or between about 40% and about 96%, or between about 40% and about 94%, or between about 40% and about 92%, or between about 40% and about 90%, between about 45% and about 99%, or between about 45% and about 98%, or between about 45% and about 96%, or between about 45% and about 94%, or between about 45% and about 92%, or between about 45% and about 90%, between about 50% and about 99%, or between about 50% and about 98%, or between about 50% and about 96%, or between about 50% and about 94%, or between about 50% and about 92%, or between about 50% and about 90%, between about 55% and about 99%, or between about 55% and about 98%, or between about 55% and about 96%, or between about 55% and about 94%, or between about 55% and about 92%, or between about 55% and about 90%, between about 60% and about 99%, or between about 60% and about 98%, or between about 60% and about 96%, or between about 60% and about 94%, or between about 60% and about 92%, or between about 60% and about 90%, of cells of the enriched heterogeneous renal cell population express OSR1.

In the methods, if the percentage of cells of the enriched heterogeneous renal cell population that express the one or more (at least one, or at least two, or at least three, or at least four, or at least five, or at least six, or at least seven, or eight) marker(s) is determined, and the one or more (at least one, or at least two, or at least three, or at least four, or at least five, or at least six, or at least seven, or eight) marker(s) is, or includes LHX1, then the enriched heterogeneous renal cell population may be identified as having therapeutic potential if at least about 0.80% of cells of the enriched heterogeneous renal cell population express LHX1. Alternatively, if the one or more (at least one, or at least two, or at least three, or at least four, or at least five, or at least six, or at least seven, or eight) marker(s) is, or includes, LHX1, then the enriched heterogeneous renal cell population may be identified as having therapeutic potential if at least about 1%, or at least about 3%, or at least about 5%, or at least about 7%, or at least about 9%, or at least about 11%, or at least about 13%, or at least about 15% of cells of the enriched heterogeneous renal cell population express LHX1. If the one or more (at least one, or at least two, or at least three, or at least four, or at least five, or at least six, or at least seven, or eight) marker(s) is, or includes LHX1, then the enriched heterogeneous renal cell population may be identified as having therapeutic potential if greater than 0% and up and up to at most 99%, or greater than 0% and up to at most 90%, or greater than 80% and up to at most about 75%, or greater than 0% and up to at most about 70%, or greater than 0% and up to at most about 65%, or greater than 0% and up to at most about 60%, or greater than 0% and up to at most about 55%, or greater than 0% and up to at most about 50%, or greater than 0% and up to at most about 45%, or greater than 0% and up to at most about 40%, or greater than 0% and up to at most about 35%, or greater than 0% and up to at most about 30% of cells of the heterogeneous renal cell population express LHX1. Further, if the one or more (at least one, or at least two, or at least three, or at least four, or at least five, or at least six, or at least seven, or eight) marker(s) is, or includes LHX1, then the enriched heterogeneous renal cell population may be identified as having therapeutic potential if between about 0.80% and about 99%, or between about 0.80% and about 90%, or between about 0.80% and about 80%, or between about 0.80% and about 70%, or between about 0.80% and about 60%, or between about 0.80% and about 50%, or between about 0.80% and about 40%, or between about 5% and about 99%, or between about 5% and about 90%, or between about 5% and about 80%, or between about 5% and about 70%, or between about 5% and about 60%, or between about 5% and about 50%, or between about 5% and about 40%, or between about 10% and about 99%, or between about 10% and about 90%, or between about 10% and about 80%, or between about 10% and about 70%, or between about 10% and about 60%, or between about 10% and about 50%, or between about 10% and about 40% of cells of the enriched heterogeneous renal cell population express LHX1.

In the methods, if the percentage of cells of the enriched heterogeneous renal cell population that express the one or more (at least one, or at least two, or at least three, or at least four, or at least five, or at least six, or at least seven, or eight) marker(s) is determined, and the one or more (at least one, or at least two, or at least three, or at least four, or at least five, or at least six, or at least seven, or eight) marker(s) is, or includes RET, then the enriched heterogeneous renal cell population may be identified as having therapeutic potential if at least about 1% of cells of the enriched heterogeneous renal cell population express RET. Alternatively, if the one or more (at least one, or at least two, or at least three, or at least four, or at least five, or at least six, or at least seven, or eight) marker(s) is, or includes, RET, then the enriched heterogeneous renal cell population may be identified as having therapeutic potential if at least about 2%, at least about 3%, at least about 4%, at least about 5%, at least about 6%, at least about 7%, at least about 8%, at least about 9%, at least about 10%, at least about 11% or at least about 12% of cells of the enriched heterogeneous renal cell population express RET. If the one or more (at least one, or at least two, or at least three, or at least four, or at least five, or at least six, or at least seven, or eight) marker(s) is, or includes RET, then the enriched heterogeneous renal cell population may be identified as having therapeutic potential if greater than 1% and up to at most about 85%, or greater than 1% and up to at most about 83%, or greater than 1% and up to at most about 80%, or greater than 1% and up to at most about 77%, or greater than 1% and up to at most about 75%, or greater than 1% and up to at most about 72%, or greater than 1% and up to at most about 70%, or greater than 1% and up to at most about 67%, or greater than 1% and up to at most about 65%, or greater than 1% and up to at most about 63% or greater than 1% and up to at most about 60% of cells of the enriched heterogeneous renal cell population express RET. Further, if the one or more (at least one, or at least two, or at least three, or at least four, or at least five, or at least six, or at least seven, or eight) marker(s) is, or includes RET, then the enriched heterogeneous renal cell population may be identified as having therapeutic potential if between about 1% and about 80%, or between about 1% and about 77%, or between about 2% and about 80%, or between about 2% and about 77%, or between about 4% and about 80%, or between about 4% and about 78%, or between about 5% and about 70%, or between about 5% and about 65%, or between about 5% and about 60%, or between about 10% and about 80%, or between about 10% and about 75%, or between about 10% and about 70%, or between about 10% and about 65% or between about 10% and about 60% of cells of the enriched heterogeneous renal cell population express RET.

In the methods, if the percentage of cells of the enriched heterogeneous renal cell population that express the one or more (at least one, or at least two, or at least three, or at least four, or at least five, or at least six, or at least seven, or eight) marker(s) is determined, and the one or more (at least one, or at least two, or at least three, or at least four, or at least five, or at least six, or at least seven, or eight) marker(s) is, or includes FGF8, then the enriched heterogeneous renal cell population may be identified as having therapeutic potential if at least about .01% of cells of the enriched heterogeneous renal cell population express FGF8. Alternatively, if the one or more (at least one, or at least two, or at least three, or at least four, or at least five, or at least six, or at least seven, or eight) marker(s) is, or includes, FGF8, then the enriched heterogeneous renal cell population may be identified as having therapeutic potential if at least about 0.01%, or at least about 0.03%, or at least about 0.05%, or at least about 0.07%, or at least about 0.09%, or at least about 0.10%, or at least about 0.20%, or at least about 0.30%, or at least about 0.40%, or at least about 0.50%, or at least about .60%, or at least about 0.70%, or at least about 0.80% or at about 0.90% of cells of the enriched heterogeneous renal cell population express FGF8. If the one or more (at least one, or at least two, or at least three, or at least four, or at least five, or at least six, or at least seven, or eight) marker(s) is, or includes FGF8, then the enriched heterogeneous renal cell population may be identified as having therapeutic potential if greater than 0% and up to at most about 60% or greater than 0% and up to at most about 57%, or greater than 0% and up to at most about 55%, or greater than 0% and up to at most about 52%, or greater than 0% and up to at most about 50%, or greater than 0% and up to at most about 47%, or greater than 0% and up to at most about 45%, or greater than 0% and up to at most about 42%, or greater than 0% and up to at most about 40%, or greater than 0% and up to at most about 37% or greater than 0% and up to at most about 35% of cells of the enriched heterogeneous renal cell population express FGF8. Further, if the one or more (at least one, or at least two, or at least three, or at least four, or at least five, or at least six, or at least seven, or eight) marker(s) is, or includes FGF8, then the enriched heterogeneous renal cell population may be identified as having therapeutic potential if between about 0.01% and about 60%, or between about 0.01% and about 58%, or between about 0.01% and about 50%, or between about 0.01% and about 45%, or between about 0.01% and about 40%, or between about 0.01% and about 35%, or between about 0.01% and about 30%, or between about 0.01% and about 25%, 0.10% and about 60%, or between about 0.10% and about 58%, or between about 0.10% and about 50%, or between about 0.10% and about 45%, or between about 0.10% and about 40%, or between about 0.10% and about 35%, or between about 0.10% and about 30%, or between about 0.10% and about 25%, 0.20% and about 60%, or between about 0.20% and about 58%, or between about 0.20% and about 50%, or between about 0.20% and about 45%, or between about 0.20% and about 40%, or between about 0.20% and about 35%, or between about 0.20% and about 30%, or between about 0.20% and about 25% of cells of the enriched heterogeneous renal cell population express FGF8.

In the methods, if the percentage of cells of the enriched heterogeneous renal cell population that express the one or more (at least one, or at least two, or at least three, or at least four, or at least five, or at least six, or at least seven, or eight) marker(s) is determined, and the one or more (at least one, or at least two, or at least three, or at least four, or at least five, or at least six, or at least seven, or eight) marker(s) is, or includes RACK1, then the enriched heterogeneous renal cell population may be identified as having therapeutic potential if at least about 75% of cells of the enriched heterogeneous renal cell population express RACK1. Alternatively, if the one or more (at least one, or at least two, or at least three, or at least four, or at least five, or at least six, or at least seven, or eight) marker(s) is, or includes, RACK1, then the enriched heterogeneous renal cell population may be identified as having therapeutic potential if at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or about 100% of cells of the enriched heterogeneous renal cell population express RACK1.

In the methods, if the percentage of cells of the enriched heterogeneous renal cell population that express the one or more (at least one, or at least two, or at least three, or at least four, or at least five, or at least six, or at least seven, or eight) marker(s) is determined, and the one or more (at least one, or at least two, or at least three, or at least four, or at least five, or at least six, or at least seven, or eight) marker(s) is, or includes nephrin, then the enriched heterogeneous renal cell population may be identified as having therapeutic potential if at least about 65% of cells of the enriched heterogeneous renal cell population express nephrin. Alternatively, if the one or more (at least one, or at least two, or at least three, or at least four, or at least five, or at least six, or at least seven, or eight) marker(s) is, or includes, nephrin, then the enriched heterogeneous renal cell population may be identified as having therapeutic potential if at least about 68%, at least about 70%, at least about 72%, at least about 74%, at least about 76%, at least about 78%, at least about 80%, at least about 82%, at least about 84%, at least about 86%, at least about 88%, at least about 90%, at least about 92%, at least about 94%, at least about 96%, at least about 98%, or about 100% of cells of the enriched heterogeneous renal cell population express nephrin. If the one or more (at least one, or at least two, or at least three, or at least four, or at least five, or at least six, or at least seven, or eight) marker(s) is, or includes nephrin, then the enriched heterogeneous renal cell population may be identified as having therapeutic potential if between about 65% and about 99%, or between about 67% and about 99%, or between about 69% and about 99%, or between about 70% and about 99%, or between about 72% and about 99%, and between about 74% and about 99%, or between 35% and about 95%, or between about 40% and about 95%, or between about 45% and 95%, or between about 50% and about 95%, or between about 55% and about 95%, or between about 60% and about 95%, or between about 65% and about 95%, or between 70% and about 95%, or between about 75% and about 95%, or between about 80% and about 95% or between about 85% and about 95% of cells of the enriched heterogeneous renal cell population express nephrin.

In the methods, if the percentage of cells of the enriched heterogeneous renal cell population that express the one or more (at least one, or at least two, or at least three, or at least four, or at least five, or at least six, or at least seven, or eight) marker(s) is determined, and the one or more (at least one, or at least two, or at least three, or at least four, or at least five, or at least six, or at least seven, or eight) marker(s) is, or includes podocin, then the enriched heterogeneous renal cell population may be identified as having therapeutic potential if at least about 90% of cells of the enriched heterogeneous renal cell population express podocin. Alternatively, if the one or more (at least one, or at least two, or at least three, or at least four, or at least five, or at least six, or at least seven, or eight) marker(s) is, or includes, podocin, then the enriched heterogeneous renal cell population may be identified as having therapeutic potential if at least about 91%, at least about 92%, at least about 93%, at least about 94%, at least about 95%, at least about 96%, at least about 97%, at least about 98%, at least about 98%, or about 100% of cells of the enriched heterogeneous renal cell population express podocin.

In the methods, if the percentage of cells of the heterogeneous renal cell population that express (i) NEPH1 and the (ii) one or more (at least one, or at least two, or at least three, or at least four, or at least five, or at least six, or at least seven, or eight) marker(s) is determined, the determined percentage of cells that express (i) NEPH1 and the (ii) one or any combination of any two, any three, any four, any five, any six, any seven or all eight of markers SIX2, OSR1, LHX1, RET, FGF8, RACK1, nephrin and podocin may identify it as having a therapeutic potential. For example, the enriched heterogeneous renal cell population may be identified as having a therapeutic potential if (a) it is determined that between about 2% and about 8.0%, between about 3.0% and about 7.0% or between about 4.0% and about 6.0% of cells of the heterogeneous renal cell population express NEPH1 and (b) a combination of any two, any three, any four, any five, any six, any seven, or all eight of the following are determined: (i) greater than 0% and up to at most about 35.0% of cells of the heterogeneous renal cell population express SIX2, (ii) at least about 35% of cells of the heterogeneous renal cell population express OSR1, (iii) at least about 1% of cells of the heterogeneous renal cell population express LHX1, (iv) at least about 1% of cells of the heterogeneous renal cell population express RET, (v) greater than 0% and up to at most about 60% of cells of the heterogeneous renal cell population express FGF8, (vi) at least about 80% of cells of the heterogeneous renal cell population express RACK1, (vii) at least about 65% of cells of the enriched heterogeneous renal cell population express nephrin, and/or (viii) about 90% of cells of the enriched heterogeneous renal cell population express podocin. In another example, the enriched heterogeneous renal cell population may be identified as having a therapeutic potential if (a) it is determined that between about 2% and about 8.0%, between about 3.0% and about 7.0% or between about 4.0% and about 6.0% of cells of the heterogeneous renal cell population express NEPH1 and (b) a combination of any two, any three, any four, any five, any six, any seven, or all eight of the following are determined: (i) between about 0.1% and about 30.0% of cells of the heterogeneous renal cell population express SIX2, (ii) between about 40% and about 99% of cells of the heterogeneous renal cell population express OSR1, (iii) at least about 1% of cells of the heterogeneous renal cell population express LHX1, (iv) between about 1% and about 80 of cells of the heterogeneous renal cell population express RET, (v) between about 0.01% and about 60% of cells of the heterogeneous renal cell population express FGF8, (vi) at least about 80% of ells of the heterogeneous renal cell population express RACK1, (vii) at least about 65% of cells of the enriched heterogeneous renal cell population express nephrin, and/or (viii) about 90% of cells of the enriched heterogeneous renal cell population express podocin. The combination of any two, any three, any four, any five, any six, any seven or all eight markers whose expression may be determined at these percentages may be a combination of any of SIX2 and OSR1; SIX2 and LHX1; SIX2 and RET; SIX2 and FGF8; OSR1 and LHX1; OSR1 and RET; OSR1 and FGF8; LHX1 and RET; LHX1 and FGF8; RET and FGF8; nephrin and SIX2; nephrin and OSR1; nephrin and LHX1, nephrin and RET, nephrin and FGF8; podocin and nephrin; podocin and SIX2; podocin and OSR1; podocin and LHX1; podocin and RET; podocin and FGF8; RACK1 and nephrin; RACK1 and podocin; RACK1 and SIX2; RACK1 and OSR1; RACK1 and LHX1; RACK1 and RET; RACK1 and FGF8; SIX2, OSR1 and LHX1; SIX2, OSR1 and RET; SIX2, OSR1 and FGF8; SIX2, LHX1 and RET; SIX2, LHX1 and FGF8; SIX2, RET and FGF8; OSR1, LHX1 and RET; OSR1, LHX1 and FGF8; OSR1, RET and FGF8; LHX1, RET and FGF8; nephrin, SIX2 and OSR; nephrin, SIX2 and LHX1; nephrin, SIX2 and RET; nephrin, SIX2 and FGF8; nephrin, OSR and LHX1; nephrin, OSR and RET; nephrin, OSR and FGF8; nephrin, LHX1 and RET; nephrin, LHX1 and FGF8; nephrin, RET and FGF8; podocin, SIX2 and OSR; podocin, SIX2 and LHX1; podocin, SIX2 and RET; podocin, SIX2 and FGF8; podocin, OSR and LHX1; podocin, OSR and RET; podocin, OSR and FGF8; podocin, LHX1 and RET; podocin, LHX1 and FGF8; podocin, RET and FGF8; RACK1, SIX2 and OSR; RACK1, SIX2 and LHX1; RACK1, SIX2 and RET; RACK1, SIX2 and FGF8; RACK1, OSR and LHX1; RACK1, OSR and RET; RACK1, OSR and FGF8; RACK1, LHX1 and RET; RACK1, LHX1 and FGF8; RACK1, RET and FGF8; nephrin, podocin and RACK1; nephrin, podocin and SIX2; nephrin, podocin and OSR; nephrin, podocin and RET; nephrin, podocin and LHX1; nephrin, podocin and FGF8; nephrin, RACK1 and SIX2; nephrin, RACK1 and OSR; nephrin, RACK1 and RET; nephrin, RACK1 and LHX1; nephrin, RACK1 and FGF8; podocin, RACK1 and SIX2; podocin, RACK1 and OSR; podocin, RACK1 and RET; podocin, RACK1 and LHX1; podocin, RACK1 and FGF8; SIX2, OSR1, LHX1 and RET; SIX2, OSR1, LHX1 and FGF8; SIX2, LHX1, RET and FGF8; SIX2, OSR1, RET and FGF8; OSR1, LHX1, RET and FGF8; nephrin, SIX2, OSR1 and LHX1; nephrin, SIX2, OSR1, and RET; nephrin, SIX2, OSR1 and FGF8; nephrin, SIX2, LHX1 and RET; nephrin, SIX2, LHX1 and FGF8; nephrin, SIX2, RET and FGF8; nephrin, OSR1, LHX1 and FGF8; nephrin, OSR1, LHX1 and RET; nephrin, OSR1, RET and FGF8; nephrin, LHX1, RET and FGF8; nephrin, SIX2, OSR1 and LHX1; podocin, SIX2, OSR1, and RET; podocin, SIX2, OSR1 and FGF8; podocin, SIX2, LHX1 and RET; podocin, SIX2, LHX1 and FGF8; podocin, SIX2, RET and FGF8; podocin, OSR1, LHX1 and FGF8; podocin, OSR1, LHX1 and RET; podocin, OSR1, RET and FGF8; podocin, LHX1, RET and FGF8; RACK1, SIX2, OSR1 and FGF8; RACK1, SIX2, LHX1 and RET; RACK1, SIX2, LHX1 and FGF8; RACK1, SIX2, RET and FGF8; RACK1, OSR1, LHX1 and FGF8; RACK1, OSR1, LHX1 and RET; RACK1, OSR1, RET and FGF8; RACK1, LHX1, RET and FGF8; nephrin, podocin, SIX2 and OSR1; nephrin, podocin, SIX2 and LHX1; nephrin, podocin, SIX2 and RET; nephrin, podocin, SIX2 and FGF8; nephrin, podocin, OSR1 and LHX1; nephrin, podocin, OSR1 and RET; nephrin, podocin, OSR1 and FGF8; nephrin, podocin, LHX1 and RET; nephrin, podocin, LHX1 and FGF8; nephrin, podocin, RET and FGF8; RACK1, podocin, SIX2 and OSR1; RACK1, podocin, SIX2 and LHX1; RACK1, podocin, SIX2 and RET; RACK1, podocin, SIX2 and FGF8; RACK1, podocin, OSR1 and LHX1; RACK1, podocin, OSR1 and RET; RACK1, podocin, OSR1 and FGF8; RACK1, podocin, LHX1 and RET; RACK1, podocin, LHX1 and FGF8; RACK1, podocin, RET and FGF8; RACK1, nephrin, SIX2 and OSR1; RACK1, nephrin, SIX2 and LHX1; RACK1, nephrin, SIX2 and RET; RACK1, nephrin, SIX2 and FGF8; RACK1, nephrin, OSR1 and LHX1; RACK1, nephrin, OSR1 and RET; RACK1, nephrin, OSR1 and FGF8; RACK1, nephrin, LHX1 and RET; RACK1, nephrin, LHX1 and FGF8; RACK1, nephrin, RET and FGF8; RACK1 nephrin, podocin and SIX2; RACK1, nephrin, podocin and RET; RACK1, nephrin, podocin and LHX1; RACK, nephrin, podocin and OSR1; RACK, nephrin, podocin and FGF8; SIX2, OSR1, LHX1, RET and FGF8; nephrin, SIX2, OSR1, LHX1 and RET; nephrin, SIX2, OSR1, LHX1 and FGF8; nephrin, SIX2, LHX1, RET and FGF8; nephrin, SIX2, OSR1, RET and FGF8; nephrin, OSR1, LHX1, RET and FGF8; podocin, SIX2, OSR1, LHX1 and RET; podocin, SIX2, OSR1, LHX1 and FGF8; podocin, SIX2, LHX1, RET and FGF8; podocin, SIX2, OSR1, RET and FGF8; podocin, OSR1, LHX1, RET and FGF8; RACK1, SIX2, OSR1, LHX1 and RET; RACK1, SIX2, OSR1, LHX1 and FGF8; RACK1, SIX2, LHX1, RET and FGF8; RACK1, SIX2, OSR1, RET and FGF8; RACK1, OSR1, LHX1, RET and FGF8; nephrin, podocin, SIX2, OSR1 and LHX1; nephrin, podocin, SIX2, OSR1 and RET; nephrin, podocin, SIX2, OSR1 and FGF8; nephrin, podocin, SIX2, LHX1 and RET; nephrin, podocin, SIX2, LHX1 and FGF8; nephrin, podocin, SIX2, RET and FGF8; nephrin, podocin, OSR1, LHX1 and RET; nephrin, podocin, OSR1, LHX1 and FGF8; nephrin, podocin, OSR1, RET and FGF8; nephrin, podocin, LHX1, RET and FGF8; nephrin, RACK1, SIX2, OSR1 and LHX1; nephrin, RACK1, SIX2, OSR1 and RET; nephrin, RACK1, SIX2, OSR1 and FGF8; nephrin, RACK1, SIX2, LHX1 and RET; nephrin, RACK1, SIX2, LHX1 and FGF8; nephrin, RACK1, SIX2, RET and FGF8; nephrin, RACK1, OSR1, LHX1 and RET; nephrin, RACK1, OSR1, LHX1 and FGF8; nephrin, RACK1, OSR1, RET and FGF8; nephrin, RACK1, LHX1, RET and FGF8; podocin, RACK1, SIX2, OSR1 and LHX1; podocin, RACK1, SIX2, OSR1 and RET; podocin, RACK1, SIX2, OSR1 and FGF8; podocin, RACK1, SIX2, LHX1 and RET; podocin, RACK1, SIX2, LHX1 and FGF8; podocin, RACK1, SIX2, RET and FGF8; podocin, RACK1, OSR1, LHX1 and RET; podocin, RACK1, OSR1, LHX1 and FGF8; podocin, RACK1, OSR1, RET and FGF8; podocin, RACK1, LHX1, RET and FGF8; nephrin, podocin, RACK1, SIX2 and OSR1; nephrin, podocin, RACK1, SIX2 and LHX1; nephrin, podocin, RACK1, SIX2 and RET; nephrin, podocin, RACK1, SIX2 and FGF8; nephrin, podocin, RACK1, OSR1 and LHX1; nephrin, podocin, RACK1, OSR1 and RET; nephrin, podocin, RACK1, OSR1 and FGF8; nephrin, podocin, RACK1, LHX1 and RET; nephrin, podocin, RACK1, LHX1 and FGF8; nephrin, podocin, RACK1, RET and FGF8; nephrin, SIX2, OSR1, LHX1, RET and FGF8; podocin, SIX2, OSR1, LHX1, RET and FGF8; RACK1, SIX2, OSR1, LHX1, RET and FGF8; nephrin, podocin, SIX2, OSR1, LHX1 and RET; nephrin, podocin, SIX2, OSR1, LHX1 and FGF8; nephrin, podocin, SIX2, LHX1, RET and FGF8; nephrin, podocin, SIX2, OSR1, RET and FGF8; nephrin, podocin, OSR1, LHX1, RET and FGF8; nephrin, RACK1, SIX2, OSR1, LHX1 and RET; nephrin, RACK1, SIX2, OSR1, LHX1 and FGF8; nephrin, RACK1, SIX2, LHX1, RET and FGF8; nephrin, RACK1, SIX2, OSR1, RET and FGF8; nephrin, RACK1, OSR1, LHX1, RET and FGF8; podocin, RACK1, SIX2, OSR1, LHX1 and RET; podocin, RACK1, SIX2, OSR1, LHX1 and FGF8; podocin, RACK1, SIX2, LHX1, RET and FGF8; podocin, RACK1, SIX2, OSR1, RET and FGF8; podocin, RACK1, OSR1, LHX1, RET and FGF8; nephrin, podocin, RACK1, SIX2, OSR1 and LHX1; nephrin, podocin, RACK1, SIX2, OSR1 and RET; nephrin, podocin, RACK1, SIX2, OSR1 and FGF8; nephrin, podocin, RACK1, SIX2, LHX1 and RET; nephrin, podocin, RACK1, SIX2, LHX1 and FGF8; nephrin, podocin, RACK1, SIX2, RET and FGF8; nephrin, podocin, RACK1, OSR1, LHX1 and RET; nephrin, podocin, RACK1, OSR1, LHX1 and FGF8; nephrin, podocin, RACK1, OSR1, RET and FGF8; nephrin, podocin, RACK1, LHX1, RET and FGF8; podocin, RACK1, SIX2, OSR1, LHX1, RET and FGF8; nephrin, RACK1, SIX2, OSR1, LHX1, RET and FGF8; nephrin, podocin, SIX2, OSR1, LHX1, RET and FGF8; nephrin, podocin, RACK1, OSR1, LHX1, RET and FGF8; nephrin, podocin, RACK1, SIX2, LHX1, RET and FGF8; nephrin, podocin, RACK1, SIX2, OSR1, RET and FGF8; nephrin, podocin, RACK1, SIX2, OSR1, LHX1, and FGF8; nephrin, podocin, RACK1, SIX2, OSR1, LHX1, and RET; or SIX2, OSR1, LHX1, RET, FGF8, RACK1, nephrin and podocin.

In the methods of identifying an enriched heterogeneous renal cell population as having a therapeutic potential, it may be determined whether cells of the enriched heterogeneous renal cell population express one or more further markers, i.e., markers other NEPH1, nephrin, podocin, RACK1, SIX2, OSR1, LHX1, RET and/or FGF8. The one or more further markers may be, or may include, one or more of lefty, nodal or CD24.

It should be understood that if the percentage of cells expressing a certain marker is provided as being a percentage of “about” a particular number e.g., about 5%, the percentage of cells need not be exactly the particular number, e.g., exactly 5%. Rather, it should be understood that if the percentage of cells expressing a certain marker is provided as being “about” a particular number, e.g., about 5%, then the percentage of cells expressing the certain marker may be within up to 10% of that particular number, e.g., between 4.5% and 5.5%.

In any of the methods of identifying an enriched heterogeneous renal cell population as having a therapeutic potential, the determining whether cells of the enriched heterogeneous renal cell population express (i) NEPH1 and the (ii) one or more markers (e.g, SIX2, OSR1, LHX1, FGF8, RACK1, RET, nephrin and/or podocin) and, optionally, (iii) one or more further markers may be a determining whether the cells of the enriched heterogeneous renal cell population express (i) NEPH1 and the (ii) one or more markers and, optionally, (iii) the one or more further markers in a nucleic acid, e.g., mRNA or miRNA, or polypeptide form. The expression of (i) NEPH1 and the (ii) one or more markers (e.g, SIX2, OSR1, LHX1, FGF8, RACK1, RET, nephrin and/or podocin) and, optionally, (iii) the one or more further markers by cells of the enriched heterogeneous renal cell population may be determined via any assay suitable for detecting presence of (i) NEPH1 and the (ii) one or more markers and, optionally, (iii) the one or more further markers. For example, if expression is determined in polypeptide form, it may be determined by assays such as Western blot, fluorescence activated cell sorting (FACS), enzyme linked immunosorbent assay (ELISA). If expression is determined in nucleic acid form, it may be determined by an assay such as, or that uses, Southern blot, polymerase chain reaction (PCR) or reverse transciptase PCR, serial analysis of gene expression (SAGE), Mass ARRAY, or fluorescence in situ hybridization (FISH). The assay may be one that employs a labeled detection reagent for determining whether (i) NEPH1 and the (ii) one or more markers and, optionally, (iii) the one or more further markers is present and/or percent of cells that express (i) NEPH1 and the (ii) one or more markers and, optionally, (iii) the one or more further markers. The labeled detection reagent may include (a) a portion that complexes, directly or indirectly, with NEPH1 and the one or more markers and/or futhrer markers and (b) a detection moiety. Non-limiting detection moieties include radioactive isotopes, e.g., ³⁵S, ¹⁴C, ¹²⁵I, ³H and ¹³¹I, colloidal gold particles, fluorescent labels, e.g., Texas Red, rhodamine, fluorescein, dansyl, Lissamine, phycocryterin, phycocyanin, SPECTRUM ORANGE, SPECTRUM GREEN1 and enzyme substrates, e.g., firefly luciferase, bacterial luciferase, luciferin, horseradish peroxidase, alkaline phosphatase, or beta galactosidase.

The enriched heterogeneous renal cell population, which may be identified as having a therapeutic potential in any of the methods, may be enriched for one or more renal cell types such as renal epithelial cells, renal tubular cells, renal tubular epithelial cells, or renal proximal tubular cells. The enrichment of the enriched heterogeneous renal cell population for these one or more of renal cell types may be a reference to the enriched heterogeneous renal cell population having a greater percentage of the one or more renal cell types than does a kidney tissue of a patient, a kidney biopsy of a patient, or an in vitro culture of cells established from a kidney tissue or kidney biopsy of a patient, (which, collectively, may be referred to as a “starting renal cell population”). A starting renal cell population, if an in vitro culture of cells established from a kidney tissue of a patient or a kidney biopsy of a patient, may be a renal cell preparation comprising dissociated cells of a kidney tissue or kidney biopsy (e.g., cells dissociated from the kidney tissue or kidney biopsy via mincing and/or enzyme digestion), that may or may not have been treated to remove red blood cells and debris. The enriched heterogeneous renal cell population, in addition to being enriched for renal epithelial cells, renal tubular cells, renal tubular epithelial cells, and/or renal proximal tubular cells, may also include other renal cell types such as glomerular cells, podocytes, collecting duct cells and/or vascular cells.

The enriched heterogeneous renal cell population may be enriched for the one or more renal cell types as a result of having been prepared from a starting renal cell population, (e.g., a kidney tissue of a patient, a kidney biopsy of a patient, or an in vitro culture of cells established from a kidney tissue or kidney biopsy of a patient), via a method that includes a separation step. The separation step may be one that separates cells of the starting renal cell population that have passaged no more than one, two, or three times, on the basis of their buoyant density. If the separation step is one that separates cells on the basis of their buoyant density, the separation step may utilize a single or multi-step continuous or discontinuous density gradient using a density gradient media such as glycerol, glucose OptiPrep, Percoll, or Ficoll-Paque. The use of such a density gradient media in this manner may result in cells of the starting renal cell population (or starting renal cell population having been passaged at most one, two or three times) separating into one or more distinguishable fractions from which cells of the enriched heterogeneous renal cell population may be distinctly identified and isolated. The distinguishable fraction(s) may be one/those in which the buoyant density of cells in the fraction(s) is greater than about 1.045 g/mL, or greater than 1.045 g/mL, or greater than or equal to 1.045 g/mL. The distinguishable fraction(s) may be one/those in which the buoyant density of cells in the fraction(s) is greater than about 1.04 g/mL, or greater than 1.04 g/mL, or greater than or equal to 1.04 g/mL, or greater than about 1.0419 g/mL, or greater than 1.0419 g/mL, or greater than or equal to 1.0419 g/mL. The distinguishable fraction(s) may be one/those in which the buoyant density is between about 1.045 g/mL and about 1.091 g/mL, or between about 1.045 g/mL and about 1.052 g/mL. Alternatively, the separation step may be one that separates cells of the starting renal cell population (or cells of the starting renal cell population that have been passaged no more than one, two or three times), on the basis of whether they express particular markers on their surface. If the separation step separates cells on the basis of their expression of particular cell surface markers, the separation step may be one that utilizes flow cytometry. The flow cytometry may sort out cells from the starting renal cell population (or starting renal cell population having been passaged at most one, two or three times) if they express particular surface markers, such as nephrin, characteristic of, e.g., renal epithelial cells, renal tubular cells, renal tubular epithelial cells, or renal proximal tubular cells, to thereby form the isolated enriched heterogeneous renal cell population.

The enriched heterogeneous renal cell population, having been prepared from a starting renal cell population (or starting renal cell population having been passaged at most one, two or three times) may be cultured under hypoxic conditions prior to the separation step. If the cells are cultured under hypoxic conditions prior to the separation step, the cells may be cultured under conditions in which the oxygen levels are less than about 20%, or less than about 15%, or less than about 10%, or less than about 9%, or less than about 8%, or less than about 7%, or less than about 6%, or less than about 5% oxygen, or less than about 4% oxygen, or less than about 3% oxygen or less than about 2% oxygen. If the cells are cultured under hypoxic conditions, the cells may be cultured under the hypoxic conditions for at least 6 hours, at least 8 hours, at least 10 hours, at least 12 hours, at least 14 hours, at least 16 hours, at least 20 hours, at least 24 hours, at least 30 hours, at least 36 hours, at least 42 hours or at least 48 hours.

In general, the preparation of an enriched heterogeneous renal cell population may be from any starting cell population, for example, an in vitro culture of cells established from a kidney tissue of a patient or a kidney biopsy of a patient. If the enriched heterogeneous renal cell population is prepared from the in vitro culture of cells established from the kidney tissue or kidney biopsy of the patient, the cells of the in vitro culture may be expanded by passaging at most one, or at most two or at most three times. Alternatively, if desired, cells of the in vitro culture of cells established from the kidney tissue or kidney biopsy may be cryopreserved and then expanded by passaging at most one, or at most two or at most three times. Once the cells have been expanded, the expanded cells may be cryopreserved. The expanded cells, whether or not cryopreserved, may then be subject to a separation step or may then be subject to hypoxic culture conditions followed by a separation step. The enriched heterogeneous renal cell population is isolatable by having performed the separation step. Once the enriched heterogeneous renal cell population has been isolated, it may be frozen and/or analyzed prior to use as a therapeutic.

If the enriched heterogeneous renal cell population is identified as having a therapeutic potential in accordance with any of the methods disclosed herein, it may be included in a pharmaceutical composition, or administered in a method of treating kidney disease in a patient in need thereof, and/or used in the manufacture of a medicament to treat kidney disease. If the enriched heterogeneous renal cell population is identified as having a therapeutic potential and is included in a pharmaceutical composition, it may formulated as a hydrogel composition or as a liquid composition. The pharmaceutical composition may, or may not, include hyaluronic acid.

If the pharmaceutical composition is formulated as a hydrogel composition, cells of the enriched heterogeneous renal cell composition may be combined with a temperature-sensitive cell-stabilizing biomaterial. The temperature-sensitive cell-stabilizing biomaterial may be a biomaterial in a gel state at certain temperatures and a liquid state at others. For example, if the biomaterial is temperature-sensitive, the biomaterial may be in a gel state at about 8° C. or below, a substantially liquid state at about ambient temperature or above, and a solid-to-liquid transitional state between about 8° C. and about ambient temperature; or a gel state at about 4° C. or below, a liquid state at about 37° C. or above, and a solid-to-liquid transitional state between about 8° C. and about 18° C.; or a gel state at about 2° C. or below, a liquid state at about 37° C. or above, and a solid-to-liquid transitional state between about 8° C. and about 18° C.; or a gel state at about 2° C. or below and a liquid state at about 37° C. or above; or a gel state at about 4° C. or below and a liquid state at about 34° C. or above; or a gel state at about 6° C. or below and a liquid state at about 32° C. or above. The temperature-sensitive cell-stabilizing the biomaterial may include, or be made up of, one or more naturally sourced or recombinant proteins or peptides. The naturally sourced or recombinant proteins or peptides may be extracellular matrix proteins of recombinant origin, or extracellular matrix sourced from kidney or another tissue or organ, or gelatin. If the temperature-sensitive cell-stabilizing biomaterial is, or includes, gelatin, the gelatin may be derived from a Type I, alpha I collagen such as porcine Type I, alpha I collagen or recombinant human Type I, alpha I collagen. If the temperature-sensitive cell-stabilizing biomaterial is, or includes, gelatin, the gelatin may present in the therapeutic composition at about 0.5% to about 1% weight per volume (w/v), or about 0.8% to about 0.9% (w/v), or about 0.75% (w/v) or about 0.88% (w/v). Cells of the enriched heterogeneous renal cell population may be dispersed throughout the biomaterial, or substantially uniformly distributed throughout the biomaterial. Cells of the enriched heterogeneous renal cell population may be formulated in the biomaterial, e.g., gelatin, such that the number of cells per mL biomaterial is about 20×10⁶ cells per mL, about 40×10⁶ cells per mL, about 60×10⁶ cells per mL, about 100×10⁶ cells per mL, about 120×10⁶ cells per mL, about 140×10⁶ cells per mL, about 160×10⁶ cells per mL, about 180×10⁶ cells per mL, or about 200×10⁶ cells per mL.

If the pharmaceutical composition is formulated as a liquid composition, the enriched heterogeneous renal cell population may be combined with any suitable liquid, e.g. appropriate cell storage or culture medium, a saline, or combinations thereof, for immediate use or for cryopreservation up until the timing of its use. If the therapeutic composition is a liquid composition, the cells of the enriched heterogeneous renal cell population may be suspended in a pharmaceutically acceptable carrier or excipient, such as saline, buffered saline, dextrose, water, polyethyleneglycol, and/or any combinations thereof. Cells of the enriched heterogeneous renal cell population may be combined with the suitable liquid, e.g., cell storage or culture medium, such that the number of cells per mL liquid is about 20×10⁶ cells per mL, about 40×10⁶ cells per mL, about 60×10⁶ cells per mL, about 100×10⁶ cells per mL, about 120×10⁶ cells per mL, about 140×10⁶ cells per mL, about 160×10⁶ cells per mL, about 180×10⁶ cells per mL, or about 200×10⁶ cells per mL.

If the enriched heterogeneous renal cell population is identified as having a therapeutic potential, the enriched heterogeneous renal cell population, or a pharmaceutical composition comprising the enriched heterogeneous renal cell population, may be administered to a patient in a method of treating kidney disease or may be for use in a method of treating kidney disease. If the enriched heterogeneous renal cell population is identified as having a therapeutic potential, the enriched heterogeneous renal cell population, or a pharmaceutical composition comprising the enriched heterogeneous renal cell population, may be used in the manufacture of a medicament to treat kidney disease. The kidney disease may be at any stage or degree of acute or chronic renal failure. It may originate in the kidney or it may secondary to another condition, e.g., heart failure, hypertension, diabetes, autoimmune disease or liver disease. Alternatively, the kidney disease may be a kidney disease arising from an acute injury to the kidney, or the result of an anomaly of the kidney and/or urinary tract. The kidney disease may further include endocrine dysfunctions such as anemia, e.g., erythropoietin-deficiency, and mineral imbalance, e.g., Vitamin D deficiency.

If the enriched heterogeneous real cell population is identified as having a therapeutic potential, then administering the enriched heterogeneous renal cell population, or a pharmaceutical composition comprising the enriched heterogeneous renal cell population, may treat the kidney disease. It may treat the kidney disease by restoring kidney function, stabilizing kidney function, improving kidney function, reducing renal fibrosis or reducing renal inflammation in a kidney of a patient in need of such treatment. The treating the kidney disease may restore mineral balance or alleviate anemia in a patient in need of such treatment. The treating the kidney disease may delay or prevent the need for dialysis, or it may delay or prevent the need for a kidney transplant in a patient in need of a treatment for kidney disease. If the treating the kidney disease delays the need for dialysis or the need for a kidney transplant in the patient, the delay may be by at least 1 year, at least 1.5 years, at least 2 years, at least 2.5 years, at least 3 years, at least 3.5 years, at least 4 years, at least 4.5 years, at least 5 years, at least 5.5 years, at least 6 years, at least 6.5 year, at least 7 years, at least 7.5 years, at least 8 years, at least 8.5 years, at least 9 years, at least 9.5 years or at least 10 years. The treating the kidney disease may be determined by observation in an improvement in the patient's serum albumin, albumin to globulin ratio (A/G ratio), serum phosphorous, serum sodium, kidney size (measurable by ultrasound), serum calcium, phosphorous:calcium ratio, serum potassium, proteinuria, urine creatinine, serum creatinine, blood nitrogen urea (BUN), cholesterol levels, triglyceride levels and glomerular filtration rate (GFR), weight, blood pressure (mean systemic blood pressure, diastolic blood pressure, or systolic blood pressure), and physical endurance performance.

If the enriched heterogeneous renal cell population is identified as having a therapeutic potential, it may be administered to a patient by any suitable administration route known in the art. For instance, the enriched heterogeneous renal cell population, or a pharmaceutical composition comprising the enriched heterogeneous renal cell population, may be systemically administered to a patient in need of treatment for kidney disease. The enriched heterogeneous renal cell population, or a pharmaceutical composition comprising the enriched heterogeneous renal cell population, may be administered at or into the kidney(s) of a patient in need of treatment for kidney disease. If the enriched heterogeneous renal cell population is administered at or into the kidney(s) of the patient in need of treatment for kidney disease, it may be administered over a single or over multiple injection(s). It may be administered via direct laparotomy, via direct laparoscopy, transabdominally, or percutaneously. The enriched heterogeneous renal cell population, or pharmaceutical composition comprising the enriched heterogeneous renal cell population, may be administered by percutaneous injection into the renal cortex of a kidney, or may be administered by inserting a guiding cannula percutaneously to puncture the kidney capsule and then injecting the enriched heterogeneous renal cell population into the kidney. The enriched heterogeneous renal cell population, or pharmaceutical composition comprising the enriched heterogeneous renal cell population, may be administered by injection into the renal cortex of one or both kidneys of the patient. The administration of the enriched heterogeneous renal cell population, or pharmaceutical composition comprising the enriched heterogeneous renal cell population, may be by two injections wherein the first injection is into the renal cortex of one kidney and a second injection is into the renal cortex of the other kidney of the patient.

The enriched heterogeneous renal cell population, or pharmaceutical composition comprising the enriched heterogeneous renal cell population, is administered, by the any suitable route, at a therapeutically effective dose. A therapeutically effective dose, or amount, for administration to the patient in need of treatment for kidney disease may include about 1-9×10⁶ enriched heterogeneous renal cell population cells per gram estimated kidney weight of the patient. A therapeutically effective amount of the pharmaceutical composition may be a dose of about 1.0×10⁶, about 2.0×10⁶, about 3.0×10⁶, about 4.0×10⁶, about 5.0×10⁶, about 6.0×10⁶, about 7.0×10⁶, about 8.0×10⁶, about 9.0×10⁶, about 2.0-7.0×10⁶, between about 4.0-7.0×10⁶, or between about 5.0×10⁶-7.0×10⁶ cells of an enriched heterogeneous renal cell population per gram estimated kidney weight of the patient.

The administration of the therapeutic composition that includes the heterogeneous renal cell population to a patient in a method of treating kidney disease, may be by first and second injections. The first and second injections may be administered between approximately 3 and 12 months apart. The first and second injections may be administered approximately 3 months apart, approximately 4 months apart, approximately 5 months apart, approximately 6 months apart, approximately 7 months apart, approximately 8 months apart, approximately 9 months apart, approximately 10 months, approximately 11 months apart or approximately 12 months apart. The first and second injections may be administered between approximately 3 and 6 months apart, between approximately 6 and 9 months apart, between approximately 9 and 12 months apart, between approximately 3 and 9 months apart, between approximately 6 and 9 months apart or between approximately 6 and 12 months apart.

Those skilled in the art will recognize, or be able to ascertain using no more than routine experimentation, many equivalents to the specific embodiments described herein. Such equivalents are intended to be encompassed by the appended claims.

All publications, patents and patent applications mentioned in this specification are herein incorporated by reference into the specification to the same extent as if each individual publication, patent or patent application was specifically and individually indicated to be incorporated herein by reference in their entirety.

### EXAMPLES

### Example 1—Selected Renal Cells (SRCs) Stabilize and Improve Renal Function, in Part, by Co-Expressing a Unique Set of Markers Involved in Kidney Development

Introduction. Selected renal cells (SRCs) are an example of an enriched heterogeneous renal cell population, enriched for renal epithelial cells. SRCs are being advanced an as autologous cell-based therapy for treatment of chronic kidney disease (CKD). A study, coupling empirical SRC gene expression data with gene ontology, was conducted to ascertain whether human SRCs, as a standalone platform, restore diseased CKD kidney function and potentially initiate neo-kidney development by, at least in part, co-expression of proteins involved in kidney development.

Methods Human SRC nodes: Proteomic analyses had indicated that human enriched heterogeneous renal cell population, SRCs, co-express eight markers, Six2, Osr1, Lhx1, Ret, Fgf8, Rack1, Nphs1 and Nphs2 (Stavas J, et al., KI Reports, in press). These eight markers were selected as nodes for conducting the analysis in this study. Co-expression data for each of the eight markers is provided in Table 1.

TABLE 1
Compiled data for Fluorescent Activated Cell Sorting (FACS) Analysis

| | Six 2 | OSR1 | LHx1 | RET | FGF8 | RACK-1 | Nephrin | Podocin |
|---|---|---|---|---|---|---|---|---|
| Number of values | 19 | 22 | 22 | 22 | 18 | 11 | 10 | 5 |
| Minimum | 0.1200 | 43.77 | 0.8700 | 1.260 | 0.01000 | 80.50 | 68.55 | 95.12 |
| 25% Percentile | 0.6200 | 65.05 | 10.92 | 2.865 | 0.2225 | 91.00 | 78.99 | 95.42 |
| Median | 1.900 | 75.72 | 32.29 | 16.53 | 1.015 | 93.70 | 89.43 | 97.46 |
| 75% Percentile | 9.500 | 93.38 | 91.93 | 56.12 | 4.403 | 99.00 | 95.47 | 97.71 |
| Maximum | 30.70 | 99.10 | 99.10 | 78.38 | 58.50 | 99.40 | 98.53 | 97.74 |
| Range | 30.58 | 55.33 | 98.23 | 77.12 | 58.49 | 18.90 | 29.98 | 2.620 |
| Mean | 5.929 | 76.88 | 44.59 | 29.18 | 6.980 | 93.63 | 86.79 | 96.74 |
| Std. Deviation | 8.207 | 17.17 | 38.01 | 28.83 | 15.50 | 5.647 | 9.861 | 1.232 |
| Std. Error of Mean | 1.883 | 3.661 | 8.104 | 6.148 | 3.652 | 1.703 | 3.118 | 0.5508 |

Methods Co-expression analysis. The eight nodes co-expressed by the SRCs, e.g., example enriched heterogeneous renal cell population, were converted into their gene symbols (homo sapien) viz. six2, osr1, Ihx1, ret, fgf8, rack1/gnb211, nphs1, and nphs2 and seeded into STRING (string-db.org, last accessed Jun. 6, 2022) and Genemania (https://genemania.org, last accessed Jun. 6, 2022) with a query for their co-expression across human tissue.

Methods Renal progenitor cell tracing: To map expression of the eight markers across cells involved in kidney development, their genes symbols were queried using Human Fetal Kidney Atlas (Human fetal Kidney Atlas—Semraulab (leidenuniv.nl), last accessed Jun. 6, 2022). Violin plots were generated to report expression level of the eight markers across up to twenty-two cell types.

Methods Human SRC interactome and biological process analyses: A human SRC interactome was built by seeding gene symbols for the eight nodes co-expressed by human SRC into Genemania and querying for genetic interactions. Biological process maps were created using ShinyGO (bioinformatics.sdstate.edu/go/http://bioinformatics.sdstate.edu/go/, last accessed Jun. 3, 2022; false discovery rate cutoff=0.05; #of pathways shown=40) and Humanbase (https://hb.flatironisntiute.org; last accessed Jun. 4, 2022; biological process gene network; default values for minimum interaction confidence of 0.1 and maximum number of genes=15).

Methods scRNA-seq to map kirrel1 expression: SRCs were submitted to scRNA-seq to map gene expression. Briefly, SRC aliquots were stained with acridine orange and propidium iodide and cells were assessed for viability, concentration, and singleness using the LUNA-FX7 Dual Fluorescence Cell Counter (Logos Biosystems). Cells were then processed using the 10x Genomics Chromium Controller and the Chromium Single Cell 3′ GEM, Library & Gel Bead Kit v3.1 Dual Index Kit (PN-1000268) following the manufacturer's user guide (https://tinyurl.com/4855859x). Briefly, approximately 5,000 cells per sample were loaded onto the Chromium Chip G with a target recovery of 3,000 cells per sample for library preparation. Single cells, reverse transcription reagents and gel beads coated with barcoded oligos were encapsulated together in an oil droplet to produce gel beads in emulsion (GEMs). Reverse transcription was performed using a C1000 thermal cycler (Bio-Rad) to generate complementary DNA (cDNA) libraries tagged with a cell barcode and unique molecular index (UMI). GEMs were then broken and cDNA was purified using Dynabeads MyOne SILANE beads (Invitrogen) prior to 12 cDNA amplification cycles. Amplified cDNA libraries were purified with SPRIselect magnetic beads (Beckman Coulter) and were quantified using an Agilent Bioanalyzer High Sensitivity DNA chip (Agilent Technologies). Fragmentation, end repair, A-tailing and double-sided size selection using SPRIselect beads were then performed. Illumina-compatible adapters were ligated onto the size-selected cDNA fragments. Adapter-ligated cDNA was then purified using SPRIselect beads. Uniquely identifiable indexes were added to this cDNA during 12 amplification cycles. The completed sequencing libraries were then purified using SPRIselect beads, visualized using the Bioanalyzer High Sensitivity DNA chip, and pooled in an equimolar ratio. Pooled libraries were sequenced on a NextSeq 2000 machine (Illumina) at the UNC High Throughput Sequencing Facility. Libraries were denatured and diluted following standard Illumina protocol, spiked with 1% PhiX sequencing control (Illumina), and sequenced on one P3 flow cell in paired-end format (Read 1:28 cycles, i7 index: 10 cycles, i5 Index: 10 cycles, Read 2:90 cycles) to a total depth of 1.2 billion read pairs passing quality filters. Demultiplexing and preliminary analysis were performed using Cell Ranger 7.1.0 with default settings, and analysis was performed using 10X Genomics' Loupe Browser.

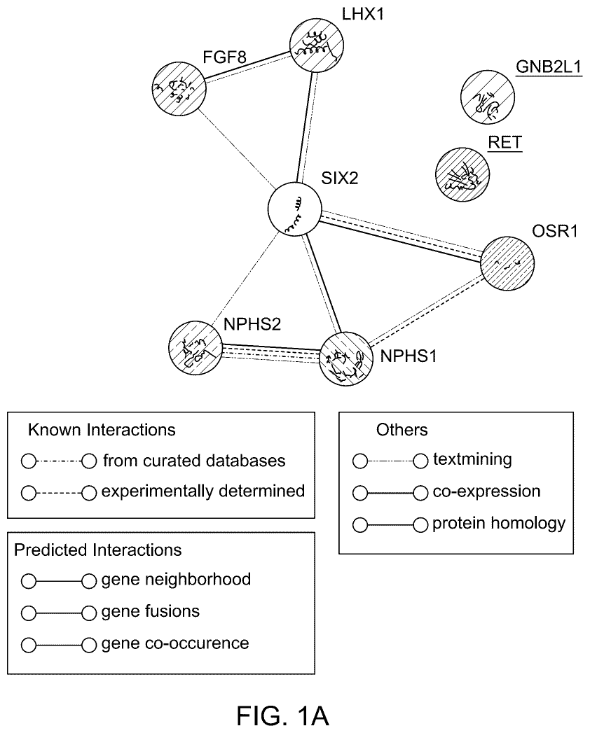

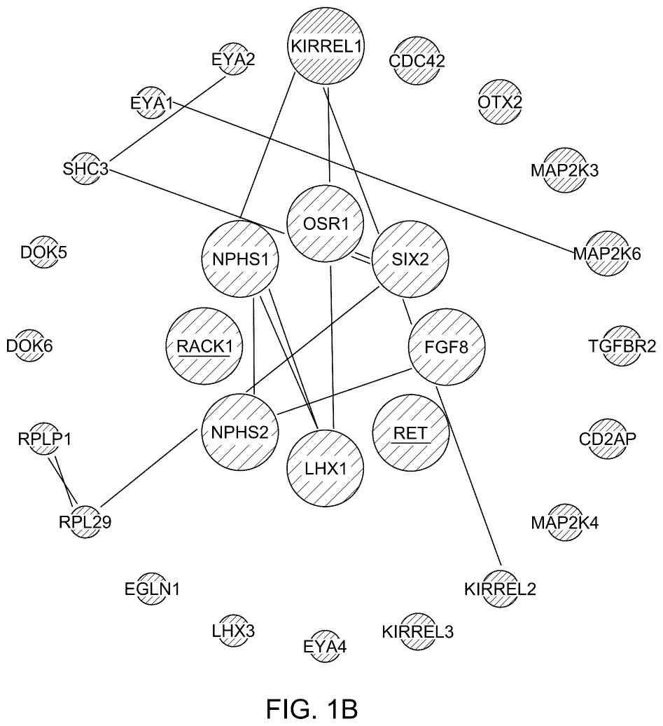

Results Profiling human SRC: Two knowledgebases, STRING and Genemania, were queried to determine whether co-expression of six2, osr1, Ihx1, ret, fgf8, rack1, nphs1 and nphs2 had been reported in human tissue. Seeding these eight genes into either database returned networks less rack1 and ret (FIGS. 1A and 1B), suggesting that all eight nodes were not known to be co-expressed human tissue.

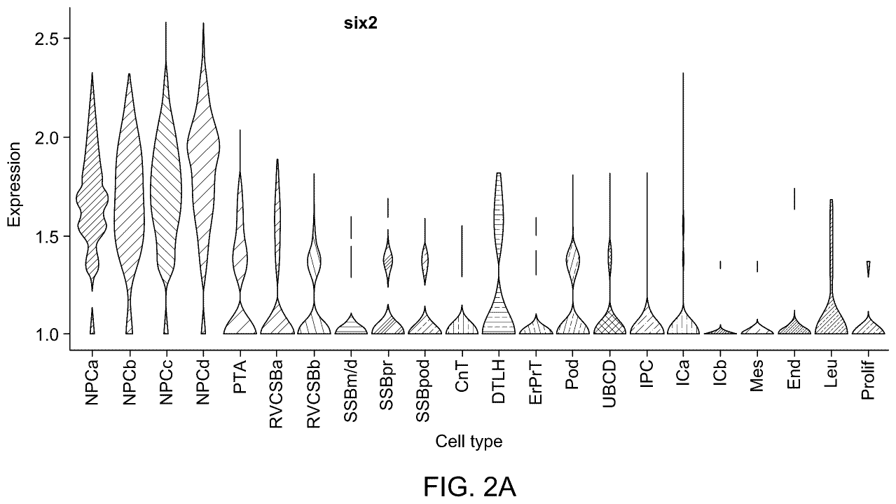

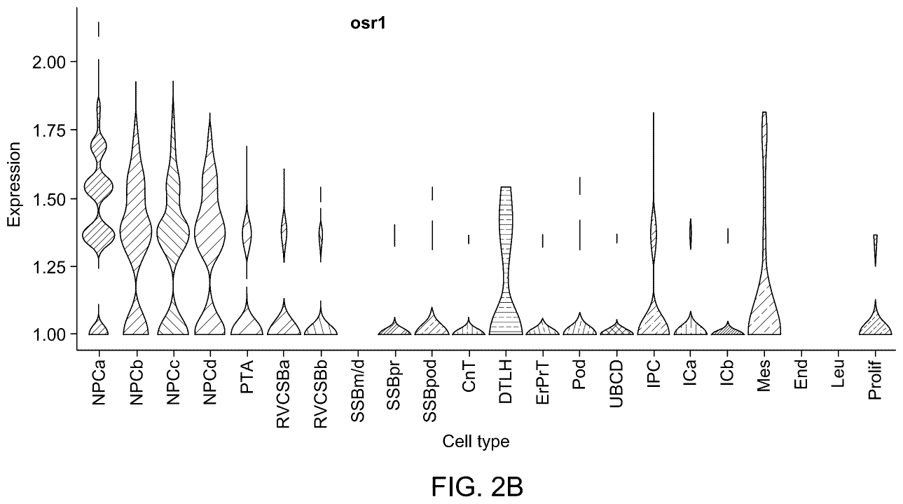

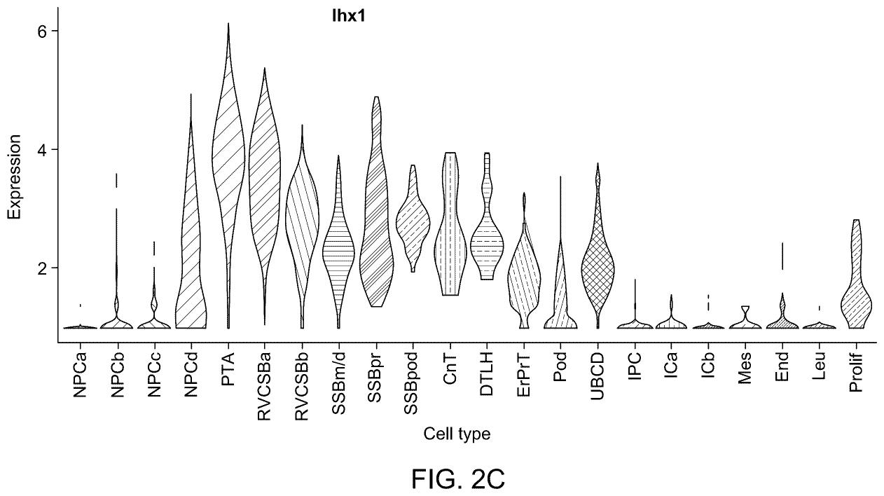

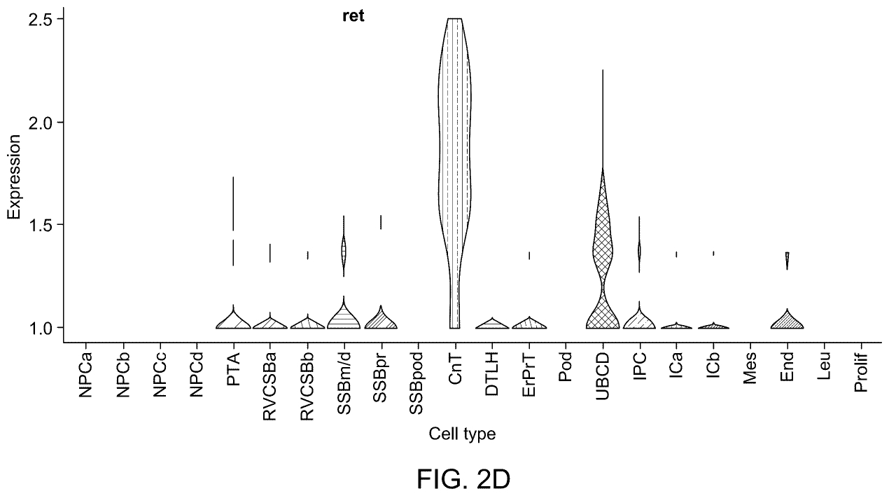

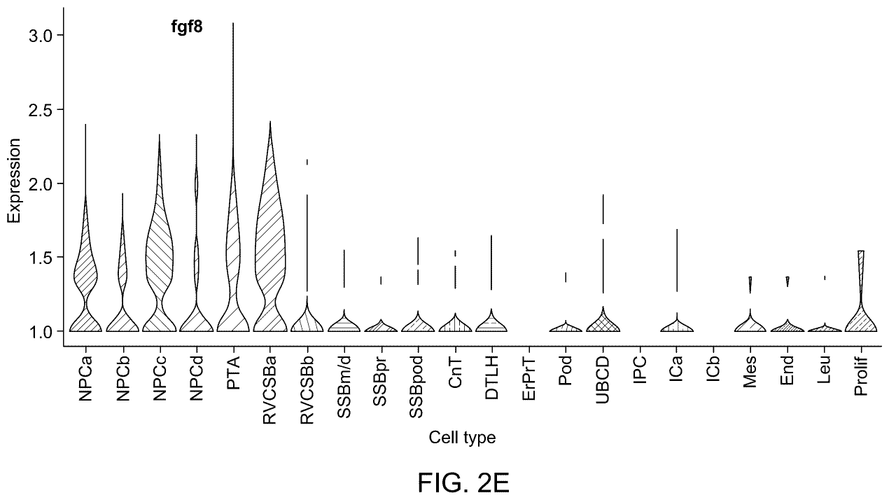

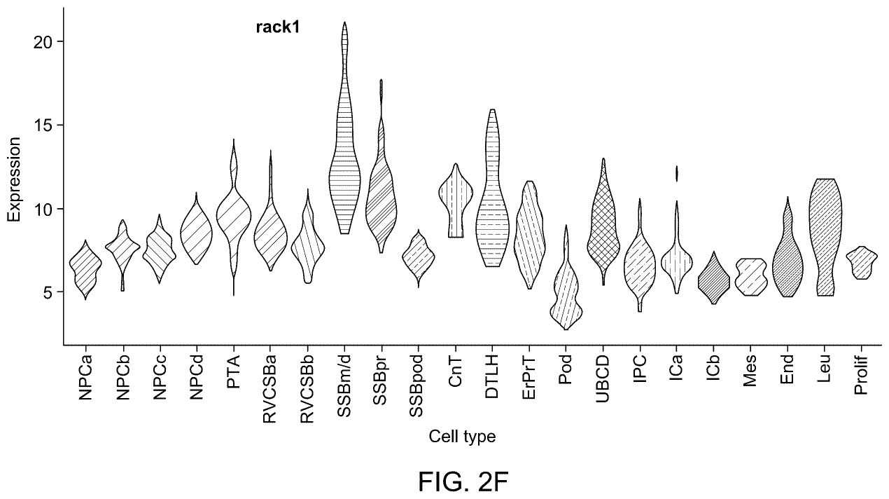

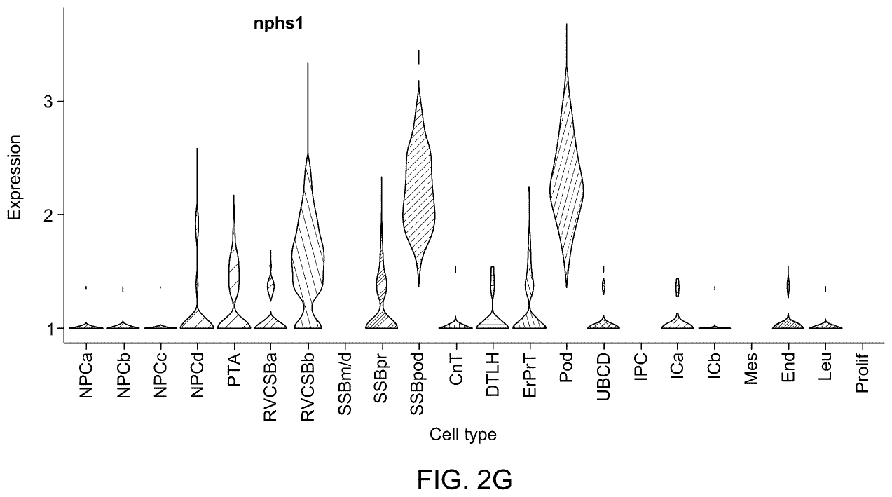

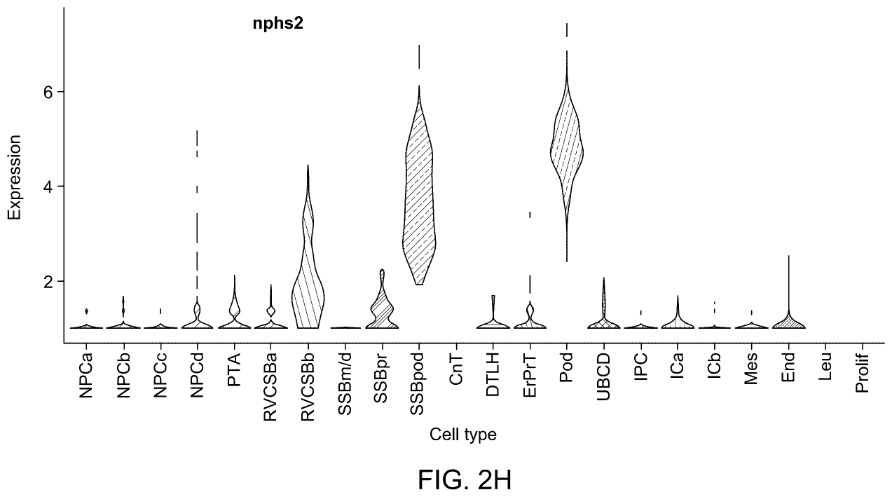

To map whether the eight markers co-expressed by the SRCs, e.g, example enriched heterogeneous renal cell population, were expressed by cells involved in early kidney development, the eight markers were queried in the Human Fetal Kidney Atlas. The Human Fetal Kidney Atlas is a repository of single-cell transcriptomics associated with twenty-two cell types involved in human kidney development. Query of this repository indicated that human SRC markers were variously expressed by a variety of renal progenitor cells including epithelial and mesenchymal progenitor pools. Genes six2 (FIG. 2A), osr1 (FIG. 2B) and Ihx1 (FIG. 2C) were predominantly expressed by nephron progenitor cells, with gene Ihx/also observed to be expressed in renal vesicle, comma-shaped body, s-shaped body, podocyte progenitor, connecting tubule, and loop of Henle cells. Expression of ret (FIG. 2D) occurred in connecting tubule cells and the ureteric bud, whereas fgf8 (FIG. 2E) expression was observed in nephron progenitor, pre-tubular aggregate and comma-shaped body cells. Rack1/guanine nucleotide-binding protein subunit beta-2-like 1 (gnb2/) (FIG. 2F) expression was relatively uniform across multiple cell types including the afore-referenced cells, and podocytes, mesangial and endothelial cells. Nphs1 (FIG. 2G) and nphs2 (FIG. 2H) were expressed on podocyte progenitors and podocytes. These findings suggested that SRCs, as a single platform, are potentially representative of the multiple renal progenitors involved in spatiotemporal patterning of the kidney.

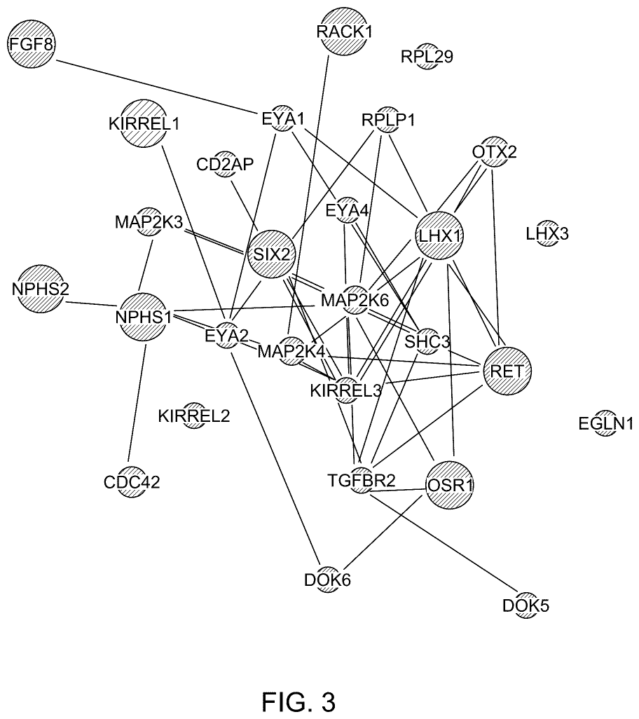

Results Human SRC interactome: Gene ontology was used to build a human SRC interactome by mapping interactions across the eight markers, e.g., nodes, co-expressed by SRC. In addition to the eight nodes, the human SRC interactome (FIG. 3) potentially included additional nodes principal among which was Kirre like Nephrin Family Adhesion Molecule 1 (kirrel 1). The kirrel 1 gene product, NEPH1, is a member of the nephrin-like protein family and interacts with podocin to maintain podocyte barrier integrity; mutations in kirrel1 are known to be associated with nephrotic syndrome (Solanki A K, et al., Kidney Int, 96 (2019): 883-889).

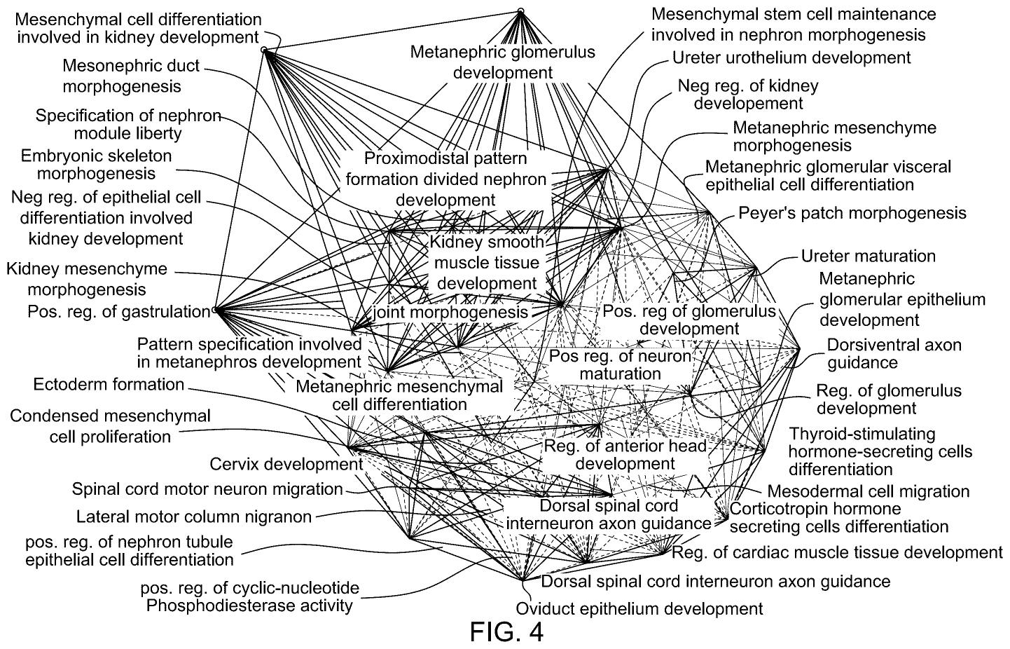

Gene ontology biological process analyses indicated that the elements within the human SRC interactome were involved in playing an integral role in all aspects of early kidney development including ureteric bud induction, mesenchymal cell differentiation, metanephric glomerulus development, pretubular aggregation and formation of comma- and S-shaped bodies, etc. (FIG. 4). The role of the elements in the human SRC interactome in these aspects, obtained in the gene ontology biological process analysis, were in agreement with the results obtained from the SRC profiling, above. That is, that SRCs, as a single platform, house elements typically expressed by renal progenitor cells.

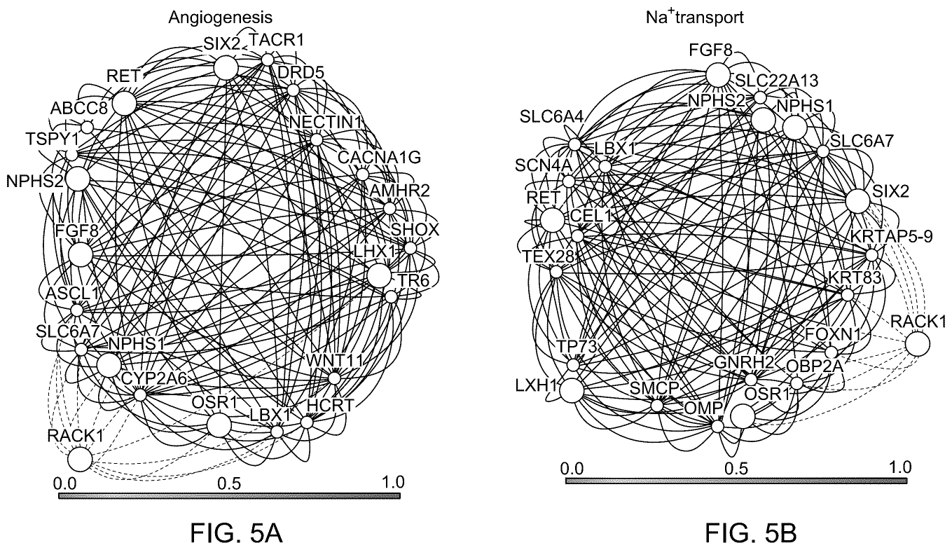

Finally, an inquiry into other renal processes likely to be associated with the human SRC interactome was conducted using Humanbase. HumanBase applies machine learning algorithms to learn biological associations from massive genomic data collections. Its integrative analyses go beyond existing biological knowledge represented in the literature to identify novel, data-driven associations. The HumanBase analysis identified angiogenesis as highly likely to be associated with the SRC interactome, as evidenced by a robust network strength for this process (FIG. 5A; network strength 0.45). Consistent with the Humanbase finding, analysis of the human SRC secretome revealed elevated VEGF-A levels (Stavas J, et al., KI Reports, in press). Na+ transport emerged as another process highly likely to be associated with the SRC network (FIG. 5B; network strength 0.74). Sodium transport in the distal and connecting tubule cells occurs through the Na⁺—Cl⁻ cotransporter which is expressed in the developing kidney, exclusively in the cells of the nascent distal tubule (Hadchouel J and Jeunemaitre X, Cell Metab 4 (2006): 335-7).

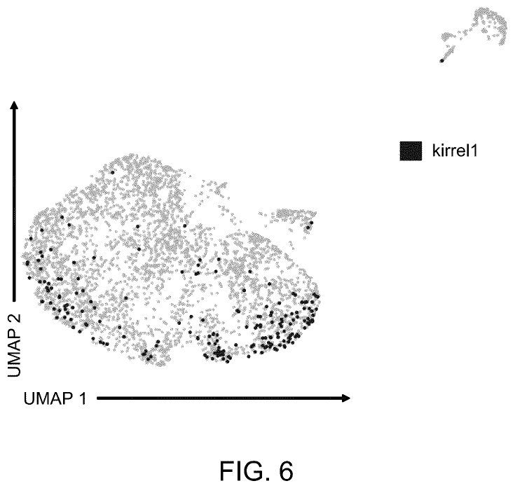

Results scRNAseq: To further confirm that SRCs include cells that express kirrel1, SRCs were submitted to scRNAseq and probed for kirrel1 expression. scRNAseq data confirmed SRC expression of kirrel1. See FIG. 6 and Table 2.

TABLE 2
Kirrell expression

| | 1 | | 2 | | 3 | | Average | | |
|---|---|---|---|---|---|---|---|---|---|
| SRC | #cells | % | #cells | % | #cells | % | #cells | % | Range % |
| kirrel1 | 127 | 5.00 | 107 | 4.00 | 196 | 6.00 | 143.33 | 5.00 | 4.00-6.00 |

SRC gene expression data has elucidated mechanisms by which SRCs may mediate regenerative and restorative activity in diseased kidneys. Expression of genes six2, osr1, Ihx1, ret, fgf8, rack1, nphs1 and nphs2 have identified SRCs as cells that may influence signaling cascades that mediate nephrogenesis or regeneration of the kidney; SRCs include cells that express genes also expressed by renal, e.g., nephron, progenitor cells. The identification of SRCs' kirrel1 expression additionally suggests SRC bioactivity may be via influence on, or involvement in, glomerular barrier function.Altogether, the empirical gene expression data coupled with the interactome analyses supported SRCs, an enriched heterogeneous renal cell population, as a standalone platform to treat CKD; supported SRCs as a therapeutic modality that, consistent with its clinical activity, possesses mechanistic capabilities for restoring endogenous repair and functional maintenance in diseased CKD kidneys, along with the potential to initiate neo-kidney development.

1. A method of identifying an enriched heterogeneous renal cell population as having a therapeutic potential, comprising:
determining whether cells of the enriched heterogeneous renal cell population express:
(i) NEPH1; and
(ii) one or more markers,
wherein the one or more markers comprises one or more of: lim homeobox protein 1 (LHX1), and fibroblast growth factor 8 (FGF8), receptor for activated C kinase 1 (RACK1) or rearranged during transfection (RET); and
identifying the enriched heterogeneous renal cell population as having therapeutic potential if cells of the enriched heterogeneous renal cell population are determined to express:
(i) NEPH1; and
(ii) the one or more markers.

2-3. (canceled)

4. The method of claim 1, wherein the one or more markers comprises LHX1.

5. The method of claim 1, wherein the one or more markers comprises FGF8.

6. The method of claim 1, wherein the one or more markers comprises RACK1.

7. The method of claim 1, wherein the one or more markers comprises RET.

8-9. (canceled)

10. The method of claim 1, wherein the one or more markers comprises at least two markers, wherein the at least two markers comprises:
(a) LHX1 and RET; (b) LHX1 and FGF8; (c) RET and FGF8; (d) RACK1 and LHX1; (e) RACK1 and RET; or (f) RACK1 and FGF8.

11. The method of claim 1, wherein the one or more markers comprises at least three markers, wherein the at least three markers comprises:
(a) LHX1, RET and FGF8; (b) RACK1, LHX1 and RET; (c) RACK1, LHX1 and FGF8; or (d) RACK1, RET and FGF8.

12. The method of claim 1, wherein the one or more markers comprises:
RACK1, LHX1, RET and FGF8.

13-19. (canceled)

20. The method of claim 1, wherein the determining comprises determining percentage of cells of the enriched heterogeneous renal population that express NEPH1.

21. The method of claim 20, wherein the enriched heterogeneous renal cell population is identified as having therapeutic potential if between about 2.0% and about 8.0% of cells of the population are determined to express NEPH1.

22-38. (canceled)

39. The method of claim 1, further comprising formulating the enriched heterogeneous renal cell population, if identified as having a therapeutic potential, in a pharmaceutical composition.

40. The method of claim 39, wherein the formulating comprises combining the cells of the enriched heterogenous renal cell population with a temperature-sensitive cell-stabilizing biomaterial.

41. The method of claim 40, wherein the temperature-sensitive cell-stabilizing biomaterial is gelatin.

42. The method of claim 39, wherein the formulating comprises combining the cells of the enriched heterogeneous renal cell population with a liquid.

43. The method of claim 42, wherein the liquid is a cell storage liquid.

44. A method of treating kidney disease in a patient, comprising:
administering the enriched heterogeneous renal cell population, identified as having therapeutic potential according to the method of claim 1, to the patient,
wherein the administering the enriched heterogeneous renal cell population to the patient treats the kidney disease in the patient.

45. The method of claim 44, wherein the treating kidney disease in the patient comprises stabilizing kidney function in the patient.

46. A method of treating kidney disease in a patient, comprising:
formulating the enriched heterogeneous renal cell population, if identified as having a therapeutic potential according to the method of claim 1, in a pharmaceutical composition; and
administering the pharmaceutical composition to the patient,
wherein the administering the pharmaceutical composition to the patient treats the kidney disease in the patient.

47. The method of claim 46, wherein the formulating comprises: combining the cells of the enriched heterogenous renal cell population with a:
(i) temperature-sensitive cell-stabilizing biomaterial; or
(ii) liquid.

48. The method of claim 46, wherein the treating kidney disease in the patient comprises stabilizing kidney function in the patient.
